(19) United States
(12) Patent Application Publication
(10) Pub. No.: US 2024/0027346 A1
(43) Pub. Date: Jan. 25, 2024

(54) DETECTION METHOD

(71) Applicant: THE UNIVERSITY OF QUEENSLAND, St Lucia, Queensland (AU)

(72) Inventors: Paul Leslie BURN, Kenmore, Queensland (AU); Paul Edward SHAW, Chelmer, Queensland (AU); Shengqiang FAN, Chapel Hill, Queensland (AU)

(21) Appl. No.: 18/253,308

(22) PCT Filed: Nov. 17, 2021
(86) PCT No.: PCT/AU2021/051362

§ 371 Date: May 17, 2023

(30) Foreign Application Priority Data
Nov. 17, 2020 (AU) 2020904243

(51) Int. Cl. G01N 21/64; G01N 31/22; C07F 7/18; C07D 235/18

(52) U.S. Cl. CPC G01N 21/6428; G01N 31/223; C07F 7/1804; C07D 235/18; G01N 2021/6439

(57) ABSTRACT

The disclosure relates to a method for detecting a nerve agent in an analyte, said nerve agent having a phosphorus-fluorine bond, which method comprises: (a) contacting the analyte with a solid state composition comprising a sensor compound; wherein the sensor compound comprises a basic nitrogen atom and a hydroxyaryl moiety protected by a silyl protecting group; and wherein, in the presence of hydrogen fluoride, said silyl group is cleaved to effect deprotection of the hydroxyl group thus forming a luminescent reporter compound; (b) irradiating the solid state composition at a predetermined wavelength; (c) measuring the luminescence to determine if the reporter compound is present; and (d) determining whether the nerve agent is present in the analyte based on the measurement obtained in step (c).

### FIELD OF THE INVENTION

This invention relates generally to the detection of nerve agents, particularly G-series nerve agents. In particular, the present invention relates to novel sensor compounds and methods for real time detection of nerve agents using a solid state composition comprising those compounds.

### BACKGROUND OF THE INVENTION

There is an on-going need to develop techniques for detection of chemical warfare agents (CWAs). In particular, there is a need for detection methods that are rapid or sensitive and have capability to detect agents at low concentrations. There is also a desire for detection methods that are selective and capable of differentiating between different classes of chemical warfare agents.

CWAs are classified into several groups, including nerve, blister, blood, choking, harassing, and incapacitation agents and toxins. In general, they were initially developed for military purposes but, given the relative ease of synthesis, they are available to terrorist groups and pose a real threat to public security. Nerve agents comprise a family of highly toxic organophosphate (OP) compounds [S. Costanzi, J.-H. Machado, M. Mitchell, ACS Chem. Neurosci. 2018, 9, 873].

Nerve agents generally enter the body through inhalation or via the skin and lead to deleterious effects on human health through interference with nerve function. Nerve agents act by inhibiting the enzyme acetylcholinesterase (AChE), which is critical for hydrolysing the neurotransmitter acetylcholine to control its concentration in the body [R. T. Delfino et al., J. Braz. Chem. Soc. 2009, 20, 407]. The inhibition of AChE can cause an accumulation of acetylcholine and result in muscle overstimulation.

Nerve agents are generally divided into two families, G and V. G-agents, including Tabun (GA), Sarin (GB), Soman (GD) and Cyclosarin (GF), were first developed before and during World War II. V-agents were synthesised later in the 1950s. A third family of nerve agents is referred to as Novichok but their molecular structures have not yet been confirmed [T. C. C. Franca et al., Int. J. Mol. Sci. 2019, 20,1222; Mirzayanov, V.S. State Secrets: An Insider's Chronicle of the Russian Chemical Weapons Program; Outskirts Press, Inc.: Parker, CO, USA, 2009; ISBN 1432725661].

G-series agents are less persistent in the environment than V-series agents. This is due to the G-series compounds being more highly volatile and more rapidly hydrolysed. As a consequence, G-series nerve agents are generally not present in a pure form. They are frequently associated with the presence of hydrogen fluoride. These considerations contribute to making their presence difficult to detect with accuracy.

Existing methods for the detection of G-series nerve agents include gas chromatography (GC), liquid chromatography (LC), ion mobility spectrometry (IMS), and Fourier transform infrared spectrometry (FTIR) [F. N. Diauudin et al., Sensing and Bio-Sensing Research, 2019, 26, 100305].

These instrument based techniques are stable and sensitive but they require expensive and bulky instrumentation and have substantial power requirements. In addition, the analytical techniques and sample preparation are complex and the instrumentation requires operation by skilled personnel; therefore these techniques do not meet the requirements for incident detection in the field.

Biosensing using enzymes, antibodies, or aptamers is an appropriate approach for on-site detection but it is limited by sensitivity or poor stability of the enzyme with respect to temperature and pH. In addition, the technique is expensive due to high enzyme production cost and difficulty for mass production [F. N. Diauudin et al. Sensing and Bio-Sensing Research, 2019, 26, 100305].

Colourimetric detection of nerve agents is another possible approach for on-site detection but this technique sometimes has the drawback of the requirement for liquid sampling in the form of liquid droplets.

A key problem with developing fluorescence-based detection of nerve agents using a chemical reaction with the sensor material is that the nerve agents themselves are not always particularly stable. For example, G-series CWAs can contain hydrogen fluoride that is present from the synthesis of the CWA or subsequent hydrolysis [S. Fan et al., “Challenges in fluorescence detection of chemical warfare agent vapours using solid-state films”, Adv. Mater., 2019, 1905785]. In this case, if the nucleophilic group required for the chemical reaction with the CWA is also basic then the nerve agent and commonly occurring acids can often interact in a similar manner. In addition, chemical reactions in the solid-state can sometimes be too slow for safe use.

There is a need for alternative methods of detecting the presence of nerve agents, such as G-series nerve agents, in the field that address one or more of the drawbacks of existing techniques. There is a desire for detection methods that have improved selectivity to differentiate from hydrogen fluoride or other acids. Increased sensitivity to detect very low levels of nerve agents or with higher accuracy is also desirable.

### SUMMARY OF THE INVENTION

The present invention relates to methods for real time detection of a nerve agent, particularly a G-series nerve agent, said nerve agent having a phosphorus-fluorine bond.

In one aspect, there is provided a method for detecting an analyte comprising a nerve agent, said nerve agent having a phosphorus-fluorine bond, which method comprises:
(a) contacting the analyte with a solid state composition comprising a sensor compound;
wherein the sensor compound comprises a basic nitrogen atom and a hydroxyaryl moiety protected by a silyl protecting group; and wherein, in the presence of hydrogen fluoride, said silyl group is cleaved to effect deprotection of the hydroxyl group thus forming a luminescent reporter compound;
(b) irradiating the solid state composition at a predetermined wavelength;
(c) measuring the luminescence to determine if the reporter compound is present; and
(d) determining whether the nerve agent is present in the analyte based on the measurement obtained in step (c).

The silyl ether sensor compound is suitably a silyl ether protected reporter compound and the reporter compound is capable of ESIPT-based luminescence upon radiation. Preferably the ESIPT reporter compound has a chromophore with a main absorption having a wavelength of greater than or equal to 350 nm.

In an embodiment, the sensor compound is a compound of Formula (IA), (IB), (IC), (ID), (IE), or (IF) as described herein.

In an embodiment, there is provided a method for detecting a nerve agent in an analyte, said nerve agent having a phosphorus-fluorine bond, which method comprises:
(a) contacting the analyte with a solid state composition comprising a sensor compound of Formula (IA):

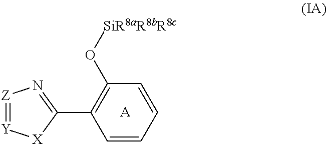

wherein:
A is an optionally substituted aryl group;
X is NR, Y is CR³, and Z is CR⁴;
X is S, Y is CR³, and Z is CR⁴;
X is O, Y is CR³, and Z is CR⁴;
X is NR, Y is CR³, and Z is NR; or
X is O, Y is CR³, and Z is N;
R is H, optionally substituted aryl or optionally substituted alkyl;
R³ is H, optionally substituted aryl or optionally substituted alkyl;
R⁴ is H, optionally substituted aryl or optionally substituted alkyl;
or R³ and R⁴, together with the carbon atoms to which they are attached, form a cyclic moiety, for example an optionally substituted aryl moiety; and
each of R^{8a}, R^{8b}, R^{8c}, which may be the same or different, is C₁₋₆ alkyl or phenyl;
or a sensor compound of Formula (TB):

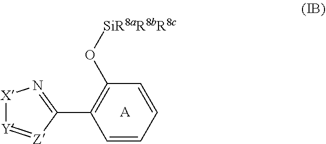

wherein:
X′ is O, Y′ is CR³ and Z′ is CR⁴;
X′ is S, Y′ is CR³ and Z′ is CR⁴; or
X′ is NR, Y′ is CR³ and Z′ is CR⁴;
and A, R, R³, R⁴, R^{8a}, R^{8b} and R^{8c} are as defined for Formula (IA);
(b) irradiating the solid state composition at a predetermined wavelength;
(c) measuring the luminescence to determine if the reporter compound is present; and
(d) determining whether the nerve agent is present in the analyte based on the measurement obtained in step (c).

It will be appreciated that the steps are not limited to the order as defined. For example, steps (a) and (b) may be simultaneous, or steps (a) to (c) or (a) to (d) may be substantially simultaneous. The irradiation step may be continuous throughout the method.

The sensor compounds as used in the methods of the invention are in the form of a solid state composition. Accordingly, in an embodiment, the sensor compound is comprised in a film or as a coating, for example a coating on a substrate. In some examples, the sensor compound is comprised on a swab. In some embodiments, the sensor compound is comprised in a sensing element, such as an optical sensing element.

In an embodiment, there is provided a method for detection of a nerve agent in an analyte, said nerve agent having a phosphorus-fluorine bond, which method comprises:
(a) irradiating an optical sensing element at a predetermined wavelength, the optical sensing element comprising a silyl ether sensor compound provided on a substrate;
wherein the sensor compound comprises a basic nitrogen atom and a hydroxyaryl moiety protected by a silyl protecting group; and wherein, in the presence of hydrogen fluoride, said silyl group is cleaved to effect deprotection of the hydroxyl group thus forming a luminescent reporter compound;
(b) contacting the analyte with the optical sensing element;
(c) measuring the luminescence of the optical sensing element; and
(d) determining whether the nerve agent is present in the analyte based on the measurement obtained in step (c).

In an embodiment, the method is for detection of an airborne nerve agent. In an embodiment, the analyte is a sample of air, particularly an air sample obtained at a location where the presence of a nerve agent containing a P—F bond is suspected, such as a battlefield or the scene of a terrorist attack. In an embodiment, the method is for detection in the gas phase or vapour phase. In an embodiment, the analyte is a soil or water sample. In an embodiment, the analyte is a surface to be tested for contamination, for example to test if decontamination of a surface has been achieved.

In an embodiment, the method is selective for nerve agents comprising a P—F bond, for example the method is selective for G-series nerve agents over commonly occurring acids, such as hydrochloric acid or acetic acid.

In another aspect, there is provided a method as described herein performed in combination with a further detection method. The methods may be carried out sequentially or simultaneously.

The methods of the invention may have application, for example, in battlefield or warfare situations, or at the scene of a suspected terrorist attack or industrial accident.

In a further aspect, the present invention also provides a sensing device comprising a solid state composition comprising a sensor compound (a solid state sensor compound) as defined herein. Accordingly, in this embodiment, the present invention provides a sensing device for detection of a nerve agent in an analyte, said nerve agent having a P—F bond, the sensing device comprising:
A solid state composition comprising a sensor compound wherein the sensor compound comprises a basic nitrogen atom and a hydroxyaryl moiety protected by a silyl protecting group; and wherein, in the presence of hydrogen fluoride, said silyl group is cleaved to effect deprotection of the hydroxyl group thus forming a luminescent reporter compound;
an irradiation source for irradiating the reporter compound with stimulating radiation at a predetermined wavelength;
a detector for measuring luminescence of the optical sensing element;
means for relating to an operator the luminescence measured by the detector; and
means for delivering the analyte for contacting with the sensor compound.

In an embodiment, the solid state composition is a sensing element. In an embodiment, the sensing device is for vapour phase detection. In an embodiment, the sensing device is for detection of a nerve agent on a surface.

In another aspect, the present invention provides a solid state composition as described herein for detection of a nerve agent comprising a P—F bond. In some embodiments, the solid state composition is a sensing element. Preferably the sensing element is for vapour phase detection.

The present inventors have discovered novel silyl ether sensor compounds having application in methods of detecting a nerve agent having a P—F bond as described herein. Accordingly, in a further aspect, the present invention provides a sensor compound of Formula (IA), (IB), (IC), (ID), (IE), or (IF), preferably a compound of Formula (IA) or Formula (IB), as described herein.

In some embodiments, the sensor compound is a compound selected from SQF1148, SQF1323, SQF1360, SQF1370, SQF1382, SQF1388, SQF1389, SQF1399, SQF13100, SQF13111, SQF1344, SQF1352, SQF1140, SQF1393, SQF1394, SQF1395, and SQF1396.

In another aspect, there is also provided a solid state composition comprising a sensor compound as described herein.

In a further aspect, there is provided a use of a sensor compound as described herein in the detection of a nerve agent, wherein the nerve agent has a P—F bond.

Although the methods, compositions and uses described herein have application in the detection of nerve agents having a P—F bond, in some preferred embodiments the nerve agent is a G-series nerve agent having a P—F bond.

### BRIEF DESCRIPTION OF THE DRAWINGS

FIG. 1 shows three methods for the generation of analyte vapour and PL measurement. Method A: Analyte was added to a 200-mL HDPE plastic bottle and kept for 30 min to allow the evaporation at 20-22° C. Sensing films were exposed to the analyte in the plastic bottle for a certain time and then moved to the optical chamber for PL measurements under ambient atmosphere. Method B: Analyte was added on the surface of a Teflon lid which was placed at the bottom of the optical chamber or a pipette droplet of sarin was added directly into the bottom of the chamber. Sensing films were placed in the same chamber for PL measurements. Method C: Analyte was added to a plastic syringe and kept for 30 min to allow the analyte evaporation. The vapour was then injected manually at a flow rate of 1 mL vapour per 3 s to a nitrogen flow (200 mL/min) and the mixed gas was introduced into the optical chamber for the sensing measurement. The nitrogen stream was eventually passed through a scrubbing solution (20 wt % sodium hydroxide in water) to break down the excess simulant.

FIG. 2 shows a series of graphical representations showing the fluorescence “turn on” at a keto-form emission wavelength (476 nm) for a film of sensor compound SQF1323 after exposing to the vapour of DFP at different times. The measurement was carried out using Method A as shown in FIG. 1. 2 μL of DFP (95% pure from ³¹P NMR) was added to a 200-mL HDPE plastic bottle and kept for 30 min to allow evaporation at 20-22° C. Sensing films were placed in the plastic bottle for a certain time and then swiftly moved to the optical chamber for PL spectra measurements under ambient atmosphere.

FIG. 3 shows a series of graphical representations of selective PL kinetics response of the films of imidazole-containing sensor compound SQF1323 to DFP vapours in comparison with acetic acid and HCl vapours. DFP gives PL “turn on” response while HCl or acetic acid does not. The measurement was carried out using Method A as shown in FIG. 1. The vapours were generated in a 200-mL plastic bottle with 2 μL of DFP (95% pure from ³¹P NMR) or 20 μL of HCl (16%) or 2 μL of acetic acid (100%) or 20 μL of acetic acid (8%).

FIG. 4 shows a graphical representation of PL kinetics response of a film of sensor compound SQF1323 to diluted DFP vapour. The measurement was carried out using Method C as shown in FIG. 1. DFP was 95% pure (³¹P NMR).

FIG. 5 shows a series of graphical representations showing the fluorescence “turn on” at a keto-form emission wavelength (525 nm) for a film of sensor compound SQF1140 after exposing to diluted DFP vapours through injection. The measurement was carried out using Method C as shown in FIG. 1.

FIG. 6 shows a series of graphical representations of PL kinetics response of the films of benzothiazole-containing sensor compound SQF1140 to the vapours of DFP, acetic acid and HCl. DFP and HCl gives PL “turn on” response while acetic acid does not. The measurement was carried out using Method C as shown in FIG. 1. Before being injected to the nitrogen carrier gas, vapours were generated in a plastic syringe (25 mL) by adding DFP (2 μL), acetic acid (2 μL) or hydrochloric acid (16%, 20 μL) and keeping for 30 min.

FIG. 7 shows a series of graphical representations of PL kinetics response of the films of benzothiazole-containing sensor compounds with various silyl ethers to repeatedly injected DFP vapours. SQF1393: R₁=R₂=methyl; SQF1394: R₁=R₂=ethyl; SQF1140: R₁=methyl and R₂=tert-butyl; SQF1395: R₁=R₂=iso-propyl; SQF1396: R₁=phenyl and R₂=tert-butyl. For SQF1393, SQF1394 and SQF1140, a sharp PL increase was observed when DFP was injected. For clarification, some arrows were added to the plot to show when the DFP was injected. The measurement was carried out using Method C as shown in FIG. 1. Before being injected to the nitrogen carrier gas, 2 μL of DFP (95%) was added to a plastic syringe (25 mL) and kept for 30 min.

FIG. 8 shows a series of graphical representations of PL kinetics response of the films of benzothiazole-containing sensor compounds to a vapour containing a real nerve agent Sarin with unknown purity. The measurement was carried out using Method B as shown in FIG. 1.

### DETAILED DESCRIPTION OF THE INVENTION

### Abbreviations

As used herein the symbols and conventions used in these processes, schemes, graphs and examples are consistent with those used in the contemporary scientific literature, for example, the Journal of the American Chemical Society. Specifically, the following abbreviations may be used in the specification: CWAs (chemical warfare agents); (ESIPT) excited state intramolecular proton transfer; PL (photoluminescence); PLQY (photoluminescence quantum yield).

### Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

By “about” is meant a quantity, level, value, number, frequency, percentage, dimension, size, amount, weight or length that varies by as much as 10, 9, 8, 7, 6, 5, 4, 3, 2 or 1% to a reference quantity, level, value, number, frequency, percentage, dimension, size, amount, weight or length. The term “approximately” is construed similarly.

When used herein the terms “w/w %”, “w/v %” and “v/v %” mean, respectively, weight to weight, weight to volume, and volume to volume percentages.

As used herein, the term “and/or” refers to and encompasses any and all possible combinations of one or more of the associated listed items, as well as the lack of combinations when interpreted in the alternative (or).

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. Thus, the use of the term “comprising” and the like indicates that the listed integers are required or mandatory, but that other integers are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Each embodiment described herein is to be applied mutatis mutandis to each and every embodiment unless specifically stated otherwise.

The terms “chemical warfare nerve agent” and “nerve agent” when used herein refer to highly toxic synthetic organophosphate compounds classed as chemical weapons. These organophosphate compounds are classed as Schedule 1 poisons. The compounds are usually dispersed in an airborne form, for example as a vapour, a mist or an aerosol. The mode of action is attack of the nervous system though inhibition of acetylcholinesterase in the body, resulting in muscle overstimulation due to buildup of the neurotransmitter acetyl choline. Exposure to nerve agents generally results in death by asphyxiation. Nerve agents are described in, for example, S. Costanzi et al., ACS Chem. Neurosci. 2018, 9, 873; T. C. C. Franca et al., Int. J. Mol. Sci. 2019, 20, 1222; and Mirzayanov, V.S. State Secrets: An Insider's Chronicle of the Russian Chemical Weapons Program; Outskirts Press, Inc.: Parker, CO, USA, 2009; ISBN 1432725661. It will be appreciated that nerve agents might not necessarily be used in, or be present in, a pure form. Typically they may be contaminated with reagents used for their synthesis, by-products of the synthesis, or decomposition or hydrolysis products resulting from reaction with molecules in the environment such as water.

Nerve agents are generally divided into two main families, namely the G-series and the V-series. A third family of nerve agents, the A-series, also exists and is commonly referred to as Novichok.

Some nerve agents contain a phosphorus-fluorine (P—F) bond. Examples of G-series nerve agents containing a P—F bond include:
Sarin (GB, propan-2-yl methylphosphonofluoridate);
Soman (GD, 3,3-dimethylbutan-2-yl methylphosphonofluoridate); and
Cyclosarin (GF, cyclohexyl methylphosphonofluoridate).

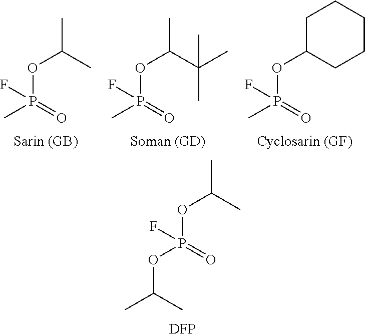

The chemical structures of the A-series compounds are reported by Mirzayanov (Mirzayanov, V.S. State Secrets: An Insider's Chronicle of the Russian Chemical Weapons Program; Outskirts Press, Inc.: Parker, CO, USA, 2009; ISBN 1432725661). Although the structures of A-series (Novichok) nerve agents are not confirmed, they are understood to have either a substituted guanidine or imidamide moiety and are reported to include the compounds A-230, A-234 and A-262 having the proposed structures below:

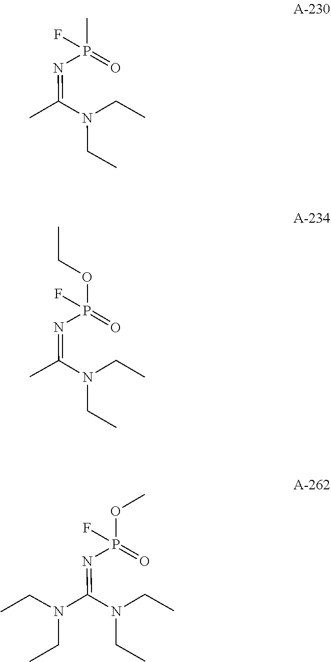

Accordingly, it is believed that these A-series nerve agents all comprise a phosphorus-fluorine (P—F) bond

Chemical weapon nerve agents are often referred to as weapons of mass destruction and, as such, production and stockpiling are restricted under the provisions of the Chemical Weapons Convention (1993). Accordingly, it is not permitted to test nerve agent detection methods under conventional laboratory conditions. For the purposes of research and initial testing, for example in the context of methods described herein, nerve agent simulants may be used to assess applicability of the methods to detection of nerve agents. Nerve agent simulants typically share some structural chemical characteristics with nerve agent molecules allowing them to be detected, but they are less toxic than the nerve agents.

Sarin, Soman and Cyclosarin are all fluorine-containing compounds and have a phosphorus-fluorine (P—F) bond. A-series compounds may also have a P—F bond. These nerve agents can all contain hydrogen fluoride (HF) as an impurity. The presence of HF can be introduced, for example as a result of the synthetic route used to prepare the nerve agent. HF may be present due to in situ decomposition of the nerve agent as a result of cleavage of the P—F bond, e.g. by hydrolysis. It will be appreciated that deliberate enhancement of the P—F cleavage could enhance the sensitivity of the detection.

In a conventional laboratory setting, nerve agent simulants may be used to test the methods described herein. Simulants for G-series nerve agents suitable for use in testing the detection methods described herein in a conventional laboratory environment include DFP (di-iso-propyl fluorophosphate). DFP may be used as a simulant of Sarin, Soman or Cyclosarin. DFP has a P—F bond and thus can also contain HF as an impurity.

Toxicity data for certain nerve agents is summarized in Table 1. LC₅₀ data (lethal concentration) is obtained from S. W. Wiener, J. Intensive Care Med, 2004, 19, 22-37. LD₅₀ data (lethal dose, g/70 kg man) is derived from T. C. C. Franca et al., Int. J. Mol. Sci. 2019, 20, 1222. LD₅₀ values for A-series nerve agents are estimated [see T. C. C. Franca et al., Int. J. Mol. Sci. 2019, 20, 1222].

TABLE 1
Toxicity Data

| NERVE | | |
|---|---|---|
| AGENT | LD₅₀ | LC₅₀ |
| Sarin | 1.7 | 1.2 ppm |
| Soman | 0.35 | 0.9 ppm |
| A-230 | 0.00075-0.002 | — |
| A-234 | 0.035 | — |
| A-262 | 0.035 | — |

It will be noted that the chemical structures of G-series and A-series nerve agents and nerve agent simulants may possess at least one chiral (asymmetric) centre. This may be an asymmetric phosphorus atom or asymmetric carbon atom(s). Soman exists as four stereoisomers due to the presence of the asymmetric P atom and the pinacolyl chiral carbon. It will be appreciated that a nerve agent may exist as a single stereoisomer, or as a mixture of isomers, including a racemate. It will also be appreciated that toxicity of different stereoisomers may be different. Isomers arising from such asymmetry (e.g., all enantiomers, stereoisomers, diastereomers, rotamers or racemates) are included within the scope of this invention. Where the stereochemistry is not specified, it will be understood that the structure is intended to encompass any stereoisomer and all mixtures thereof.

### DETAILED DESCRIPTION OF THE INVENTION

In accordance with the present invention the presence of a nerve agent having a P—F bond can be detected based on the luminescent response of a solid state composition comprising a sensor compound as described herein. The solid state composition exhibits a characteristic “turn on” photoluminescent (PL) response in the presence of nerve agent containing a P—F bond. The turn on response is believed to be elicited by the presence of hydrogen fluoride impurity in the nerve agent. In some embodiments, the nerve agent is a G-series nerve agent.

Hydrogen fluoride is a common impurity in G-series nerve agents and other nerve agents comprising a P—F bond as well as in nerve agent simulants comprising a P—F bond, such as DFP. The inventors have discovered that the hydrogen fluoride impurity can react quickly to cleave the silyl ether bond of a sensor compound of the invention to effect deprotection and provide a reporter compound with a hydroxyaryl moiety. Upon excitation the reporter compound is capable of undergoing excited-state-intramolecular-proton transfer to the keto form (enol-keto tautomerism). The keto tautomer can then decay radiatively to emit light before undergoing tautomerism back to the enol form. This process is defined as ESIPT-based luminescence. This process is sometimes referred to as ESIPT fluorescence. The reporter compound can therefore be termed as an ESIPT reporter compound. The presence of a nerve agent having a P—F bond, such as Sarin, Soman or Cyclosarin, can thus be detected based on the luminescent response of an optical sensing element comprising a sensor compound as described herein. More specifically, the fluorescent enol tautomer thus formed following desilylation can be photoexcited thereby causing a characteristic fluorescent emission from the keto tautomer. Given that hydrogen fluoride (HF) is not normally found in the environment, the characteristic PL can be detected and relied upon as an indicator for the presence of a G-series nerve agent or other nerve agent containing a P—F bond.

In the context of the present invention, what is meant by “turn on” response is that on exposure to hydrogen fluoride the sensor compound reacts with the hydrogen fluoride leading to the formation of a reporter compound. The reporter compound absorbs stimulating radiation at a predetermined wavelength and exhibits a luminescent response that can be detected at a specified wavelength. The sensor compound itself does not emit at the same wavelength as the keto tautomer of the ESIPT reporter compound and therefore its luminescent response upon photoexcitation, if any, can be ignored. Thus the method provides for greater detection sensitivity as it avoids background luminescence from the sensor compound. The presence of hydrogen fluoride can therefore be determined based on whether the “turn on” luminescent response is detected at the specified wavelength.

More specifically, in some embodiments, the sensor compound comprises a basic nitrogen atom and a hydroxyaryl moiety wherein the hydroxyaryl oxygen is protected by a silyl ether. In some embodiments, such as a sensor compound of Formula (IA) or (IB), the basic nitrogen atom is located in a 1,5-configuration relative to the position of the hydroxyaryl oxygen atom. This 1,5-configuration provides a desirable 6-membered transition state for keto-enol tautomerism. However, it will be understood that sensor compounds having other configurations can also have application in the methods of the present invention. In another embodiment, such as a sensor compound represented by the Formula (ID) as defined herein, the basic nitrogen atom is located in a 1,6-configuration relative to the position of the keto-enol oxygen atom, thus providing a 7-membered transition state.

In some embodiments, the basic nitrogen atom is comprised in a five-membered heteroaryl moiety, for example an imidazole, a pyrazole, a triazole, an oxadiazole, an oxazole, a thiazole, an isoxazole or an isothiazole in accordance with the sensor compounds as represented by the Formulae (IA) and (IB). In some embodiments, the basic nitrogen atom is comprised in a six-membered heteroaryl moiety in accordance with the sensor compounds as represented by Formula (IC) as defined herein. The basic nitrogen is not limited to cyclic basic nitrogen atoms. In some embodiments, it will be understood that the basic nitrogen atom can form part of an acyclic moiety. Non-limiting examples of sensor compounds comprising an acyclic basic nitrogen atom include azine compounds of Formula (IE) or imine compounds of (IF) as defined herein.

Without being bound by theory or mode of operation, it is believed that the compounds of the present invention react with HF present as an impurity. The HF can be introduced during synthesis of a nerve agent having a P—F bond. Alternatively, the HF can be introduced due to decomposition, for example as a result of hydrolysis of the P—F bond due to atmospheric moisture or deliberately using accelerating means such as catalysis.

Specifically, the basic nitrogen atom interacts with the HF enabling it to cleave the silyl ether causing deprotection, to generate an enol group after proton transfer. Upon photoexcitation, proton transfer in conjunction with enol-keto tautomerism occurs to give the keto form. The keto form of the reporter compound then decays to the ground state with the emission at a longer wavelength than either the sensor compound, or reporter compound in its enol form. Measurement of the PL intensity at the wavelength the keto form of the reporter emits enables the avoidance of background fluorescence affecting the detection event. That is while sensor and reporter in the enol form can be excited at the same wavelength the PL from the keto form of the reporter molecule can be selectively detected.

A proposed mechanism of the reaction sequence is summarized in the exemplary scheme below:

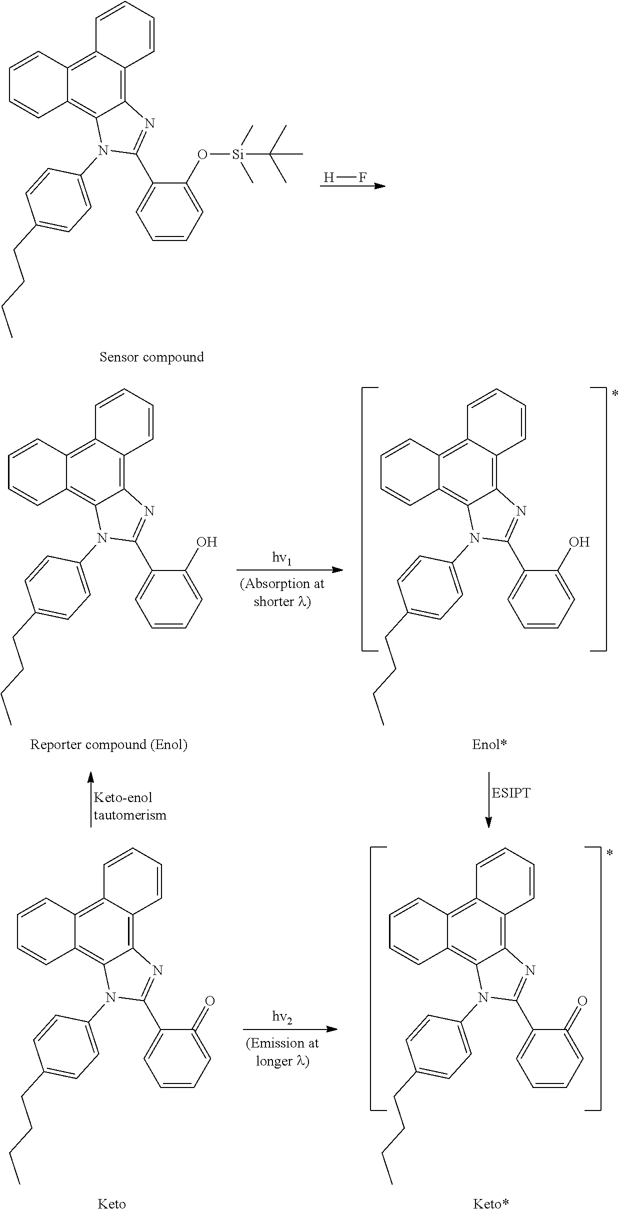

Thus, it has been discovered that sensor compounds described herein, such as the silyl ethers of Formulae (IA), (IB), (IC), (ID), (IE) and (IF) lead to a photoluminescence (PL) turn on response at the wavelength of the emission of the keto form of the reporter compound in the presence of the G-series nerve agent simulant DFP. The compounds of the invention are thus considered to find application in the detection of nerve agents having a P—F bond. In particular, these silyl ether sensor compounds offer access to real time detection.

It has been observed that the ability to achieve selectivity and the speed of the detection process is dependent on both the nitrogen base used and the silyl protecting group.

The choice of the silyl group is important in terms of the speed of response and selectivity. For example, when the silyl substituents (R^{8a}, R^{8b}, R^{8c}) are all methyl, then a rapid turn on is observed in the presence of different acid vapours and there is no selectivity for HF, and thus no detection for nerve agents comprising a P—F bond. If R^{8a}, R^{8b}, and R^{8c} are all iso-propyl, or R^{8a} and R^{8b} are phenyl and R^{8c} is t-butyl, the turn on response has been observed to be slow. Where R^{8a} and R^{8b} are methyl and R^{8c} is t-butyl, or R^{8a}, R^{8b}, and R^{8c} are all ethyl, the turn on response rate is good and selectivity is then dependent on the structure of the nitrogen base.

The choice of the nitrogen base can thus provide control over the selectivity towards HF and potential interferent acids when R^{8a}, R^{8b} are methyl and R^{8c} is t-butyl or R^{8a}, R^{8b}, R^{8c} are all ethyl. In some embodiments, the sensor compounds, for example the compounds of Formula (IA) and (IB) are selective for detection of HF present in G-series nerve agents such as Sarin, Soman and Cyclosarin. The imidazole-based compounds of Formula (IA) have been discovered to display no PL turn-on response in the presence of vapours of commonly occurring acids such as hydrochloric (HCl) or acetic acid (AcOH). The PL response in the presence of an imidazole compound of Formula (IA) is shown in FIG. 3, which demonstrates a luminescence turn on in the presence of HF and no change with vapours of the two acids.

The basic N-atom of the imidazole thus results in no PL response after protonation by acid false positives such as HCl and acetic acid.

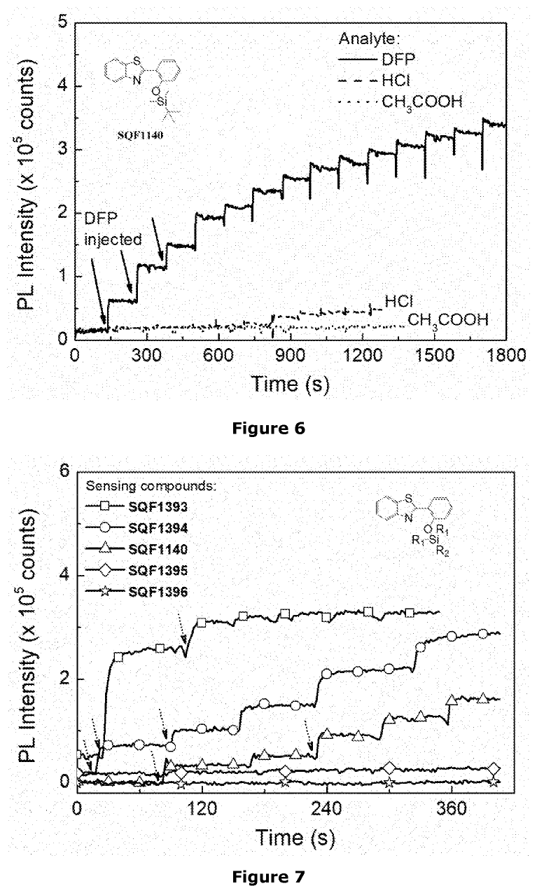

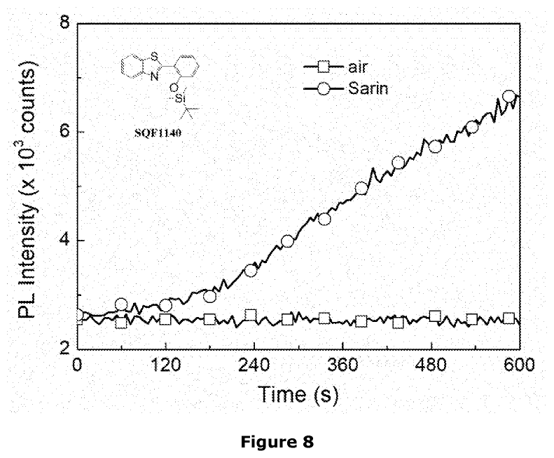

Benzothiazole-based sensor compounds wherein R^{8a} and R^{8b} are methyl and R^{8c} is t-butyl, or R^{8a}, R^{8b}, and R^{8c} are all ethyl, have been shown to display PL turn on in the presence of HCl and display no PL response to AcOH (FIG. 6). Benzothiazole-based compounds provide a very sharp PL turn on and show a fast response to HF but also have a turn on to HCl, but not acetic acid.

It has been discovered that this form of nerve agent detection has particular application for detection of airborne nerve agents, for example in the form of a vapour, mist or aerosol, although it is not so limited. Although the detection of the analyte in a vapour or aerosol phase is preferred, the analyte sample form may be different. For example, the sample may be a solid or liquid. Thus, it will be appreciated that the detection methods herein may also be applicable to detection of nerve agents in different sample forms, such as droplets or particles. Similarly, samples may extend to detection of a contaminated surface, soil samples and the like. For example, a soil sample that may be contaminated may be collected on a swab which is then placed in a heated swab head to release the vapours to enable detection. Surfaces may be sampled using similar techniques.

In another aspect of methods for detecting a nerve agent comprising a P—F bond, a surface may be tested for contamination by contacting it with a solid state composition comprising a sensor compound in the form of a substrate, for example, fabric or paper impregnated with a sensor compound as described herein. Suitably, the substrate is impregnated by soaking it in a solution of a sensor compound in a volatile solvent. The solvent is then allowed to evaporate to provide the impregnated substrate. Alternatively, the surface to be tested may be sprayed with a solution of the sensor compound in a volatile solvent to check for contamination. Evaporation of the solvent will provide a solid state sensor compound on the surface to be tested. The sensor compound can then be utilised to detect the presence of a nerve agent on the surface. It will be understood that, with respect to these embodiments, an external excitation source and fluorescence detector, which could be separate units, or combined in a single unit, could be used to ascertain whether the reporter compound had formed, thus indicating the presence of a nerve agent containing a P—F bond as a surface contaminant.

It will be appreciated that preferably the detection methods described herein operate through the detection of airborne nerve agent, such as a nerve agent in vapour, mist or aerosol form. Known methods for detection of nerve agents generally rely on liquid sampling. The present methods have an advantage of not requiring liquid sampling. Furthermore, the methods described herein can operate in real time. Thus, they can provide a fast response, measured in seconds, thus providing access to detection methods with increased convenience, ease of use, speed and safety.

In some embodiments, the detection methods described herein are believed to be capable of detecting nerve agents in concentrations of less than 100 ppm; less than 1 ppm; less than 500 ppb; or less than 250 ppb.

The detection methods may be used to detect the presence of a nerve agent comprising a P—F bond, for example a G-series agent such as Soman, Sarin or Cyclosarin. Although not so limited, in some embodiments, the nerve agent is airborne, for example in the form of a vapour, mist or aerosol. In an embodiment, the nerve agent is in the form of a vapour.

For use in the methods of the invention, those skilled in the art will understand that a sensor compound may be processed or formulated in accordance with the requirements of its intended use. The sensor compound is used in a solid state form in the form of a solid state composition. In an embodiment, the sensor compound is provided as an optical sensing element. Preferably, the sensor compound is provided as a coating or film on a substrate.

In a further aspect, the present invention provides an optical sensing element for vapour phase detection of a nerve agent comprising a P—F bond, the optical sensing element comprising a sensor compound as defined herein.

In some embodiments, the optical sensing element is for detection of airborne nerve agents, for example nerve agents in the form of a vapour, aerosol or mist.

The present invention also provides a sensing device in which the optical sensing element would be used. Accordingly, in this aspect the present invention further provides a sensing device for detection of a nerve agent in an analyte, the sensing device comprising:
an optical sensing element as described herein;
an irradiation source for irradiating the optical sensing element with stimulating radiation;
a detector for measuring luminescence of the optical sensing element;
means for delivering the analyte for contacting with the optical sensing element; and
means for relating to an operator the luminescence measured by the detector.

In some embodiments, the sensing device is adapted for detection of airborne nerve agents. In some embodiments, the sensing device is for vapour phase detection or adapted for vapour phase detection. In some embodiments, the sensing device has a swab capability for introducing the analyte sample to be tested.

The detection of a P—F containing nerve agent analyte may suitably include multiple sensor compounds, multiple sensing elements or multiple sensing techniques. The sensing agents used in this invention may be used as key components in a sensor array for selective detection. It will be appreciated that the detection methods described herein may be combined with another technique, such as a colorimetric method or a biosensor to form a binary sensing system.

For example, the compounds of the present invention may be used in conjunction with a colorimetric sensor material that responds to the presence of an organophosphate with a colour change.

The reporter compounds useful in methods of the present invention suitably have an absorption at a wavelength of 350 nm or greater. The keto tautomer of the reporter compound useful for the invention emit light at a longer wavelength than the sensor compound and the enol form of the reporter compound.

In an embodiment of the invention, in the optical sensing element the sensor compound is provided as a thin film coating on a solid transparent substrate. It will be understood that the term “transparent” refers to the ability of the substrate to allow transmission of electromagnetic radiation at the predetermined wavelength being used for excitation. In this embodiment the optical sensing element is therefore a solid-state system. The sensor compound will typically be provided as a continuous layer or coating on transparent substrate. To produce the coating the sensor compound may be dissolved in a solvent and cast or deposited on the substrate from solution. The solvent is then removed leaving the compound as a film or coating on the substrate. Examples of suitable solvents that may be useful in practice of the invention include toluene, chlorinated solvents such as dichloromethane, chloroform, acetone, ethanol, methanol, iso-propanol, tert-butanol, methoxyethanol, tetrahydrofuran (THF), N,N-dimethylformamide (DMF), dimethyl sulfoxide (DMSO), 1,3-dioxane and 1,4-dioxane, or a mixture thereof.

Determination of a suitable solvent (or solvent combination) to form a coating solution comprising a sensor compound is well within the skill and knowledge in the art. In some examples, the film or coating is deposited from toluene solution.

In another aspect, the present invention thus provides a coating or film comprising a sensor compound as defined herein.

The sensor compound may be formulated or processed in the presence of any other material such as binders, plasticisers, polymeric matrices, host matrices, and the like. In the optical sensing element, a polymer may also be employed to make a coating together with the sensor compound and substrate in particular in the scenarios that require large-area and/or thick coatings. Examples of suitable polymers that may be useful in the invention include polyethylenimine (PEI), polyethylene oxide (PEO) and cellulose acetate. Methods of coating a substrate are well known in the art and may be selected in accordance with the particular application and circumstances. Methods will depend on the shape, configuration and/or chemical composition of the substrate. Examples of methods of coating or casting films include, for example, spin-coating, blade coating or hand coating using, for example, a K bar. Other examples include ink-jet printing or spray deposition.

The amount of sensor compound provided in the coating will be that required to produce a detectable luminescent response when the sensor compound is exposed to a nerve agent. The amount of sensor compound to be included in the coating may be determined experimentally in accordance with general knowledge in the art and the teaching of the Examples herein.

The minimum amount of sensor compound provided in the coating will be that required to produce a detectable fluorescent emission from the ESIPT reporter compound when the sensor compound is exposed to HF in a nerve agent comprising a P—F bond. The amount of sensor compound included in the coating may be determined experimentally. Determination of the amount of sensor compound will be well within the skill and knowledge of the person skilled in the art.

Typically the film coating will have a thickness of 100 nm, or less. In some embodiments, the film coating has a thickness of 10 nm to 100 nm or 50 nm to 100 nm, for example 50 nm to 80 nm. In some embodiments, the coating is a thin coating of 20 nm to 50 nm, for example 20 nm to 30 nm, or 25 nm to 35 nm.

Examples of suitable substrates are well known in the art and will depend on the application. The nature of the substrate may depend on the phase employed in the method of detection. Methods of detection include those where the analyte is airborne and in the form of a vapour, aerosol or mist. It will be appreciated that the nature of the substrate should be compatible with the detection technique with regard to, for example, resistance to solubility, chemical compatibility with the sensor compound and/or analyte and/or degradation by heat, light or chemical reaction. For example, a substrate may be a glass, such as borosilicate glass, or fused silica. In some embodiments, the substrate may be formed from a plastic, for example a polycarbonate.

It will be appreciated that methods for detection of airborne analytes will be of particular use in the field of detection of nerve agents, for example on a battlefield or other warfare situation, or at the scene of a terrorist attack, suspected terrorist attack or laboratory accident. For airborne analyte detection, such as vapour phase detection, a substrate may take the form of, for example, a tube or a surface in an enclosed channel with the sensor compound provided as a coating on an internal surface of the tube or surface in the channel. In this case a sample of analyte to be tested is provided to the interior of the tube or enclosed channel for contacting where it will come into contact with the sensor compound. Where the substrate is a tube it may be a capillary tube made of a glass, such as a borosilicate glass, or fused silica. Typically the capillary tube will have an internal diameter of up to 1 mm. The length of the capillary tube is usually no more than 100 mm. Capillary tubes useful in the invention are commercially available and may be cut to an appropriate length. Alternatively, the sensor compound may be deposited onto a flat surface such as a glass slide.

A desirable property of the optical sensing element of the invention is that it is non-scattering when irradiated, as takes place during the detection process. Preferred substrates are transparent. However, in certain applications and configurations reflective substrates are also useful.

The methods of the present invention use an irradiation source for irradiating the optical sensing element with stimulating radiation in order to photoexcite the ESIPT reporter compound formed through contact of the sensor compound with an analyte sample that may include a P—F bond containing nerve agent to be detected. The optical sensing element may be irradiated continuously or may be irradiated with pulsed radiation.

The stimulating radiation is at a predetermined wavelength determined with regard to a wavelength at which the reporter compound (formed by deprotection of the sensor compound with HF) absorbs radiation and has a detectable emission from the keto form. This is the basis upon which the present invention facilitates detection of hydrogen fluoride, and thus G-series nerve agents such as Sarin, Soman and Cyclosarin or other nerve agents having a P—F bond, such as an A-series nerve agent.

Whether a particular sensor compound leads to the “turn on” response required to be useful in the present invention may be determined by analysing the optical properties of the sensor compound and the reporter compound at a particular wavelength of irradiation. This involves identifying one or more wavelengths at which the reporter compound can be excited and exhibits a detectable luminescent response. If the “turn on” response is observed for a number of different wavelengths, it may be necessary to select the wavelength based on the intensity of the “turn on” response. It will also be necessary to consider the types of irradiation source available and the wavelength capable of being supplied by the source.

For the methods described herein, generally the exciting radiation is in the near UV-deep blue or blue register. That wavelength is a wavelength at which the ESIPT reporter compound (formed by removal of the silyl ether protecting group of the sensor compound with HF associated with the nerve agent to form the enol) absorbs radiation and has a detectable emission from the keto form. A preferred wavelength is a wavelength of greater than or equal to 350 nm.

Typically, the irradiation source will be a narrowband light source such as a light-emitting diode (LED) or laser. It will also be relevant to consider the type of detector used and its detection sensitivity.

A detector is used for measuring luminescent response of the optical sensing element after exposure to a sample. It is envisaged that the luminescent response will be measured with a broadband detector such as a photodiode. To maximize sensitivity an amplified detector such as an avalanche photodiode or photomultiplier tube may be used. Alternatively, a spectrally resolved detector such as a CCD spectrograph may be used to resolve changes in the luminescence shape and intensity. In addition, a long-pass or band-pass optical filter could be included between the sensor and the detector to block the excitation wavelength from reaching the detector. The detection will include some means for relating to an operator the luminescence measured by the detector. This means may involve some form of signal, for example a signal that is communicated visually, audibly or stimulatorily (for example by vibration).

A device of the invention will also include a means for delivering an analyte sample to be analysed for contacting with the optical sensing element. For detection of airborne analyte, the sample will be in a form such as a vapour, aerosol or mist. Typically, this means a fan or blower or pump, optionally coupled with a flow meter, will be needed to continuously draw the sample into contact with the optical sensing element. For solution phase detection, the sample will be in solution and typically the sample may be drawn into contact with the sensing element using a pump. In the case the a solid sample such as soil, a swab can be used to collect the sample and then the sensing device will provide a means of releasing the analyte from the solid sample in the form of a vapour. In the case where the sensor compound is sprayed onto a potentially contaminated surface the excitation source and detector may be in different devices.

It will be appreciated that the methods described herein may be combined with an additional method to reinforce evidence for the presence of a nerve agent, particularly a G-series nerve agent. Examples of additional detection methods for use in combination with the methods described herein include commercially available colorimetric detection paper [V. Pitschmann, et al., Chemosensors 2019, 7, 30] or a commercially available biosensor [L. Matĕjoyský, V. I. Pitschmann, Biosensors 2018, 8, 51]. An additional detection method which may be used in combination with the sensor compound described herein is a secondary fluorescent sensor compound which can specifically detect organophosphonate/organophosphate functionality.

It will be understood that when a detection method as described herein is used in combination with another detection method, this combined methodology will serve to reinforce or confirm the presence of a nerve agent containing a P—F bond. It will be appreciated that detection methods may be used sequentially or simultaneously, and in any order. In an embodiment, a combined method may be carried out simultaneously using an array of sensors.

Compounds of the Invention

Sensor compounds of the invention are silyl ether derivatives of a hydroxyaryl reporter compound. The ESIPT reporter compound comprises a hydroxylaryl, such as a hydroxyphenyl, moiety and a basic nitrogen atom. The silyl group acts as a protecting group, preventing enol-keto tautomerism upon photoexcitation occurring until the silyl group is cleaved by hydrogen fluoride. Cleavage of the silyl ether provides the corresponding de-protected hydroxyaryl compound that can then undergo enol-keto tautomerism on irradiation.

In some embodiments, the ESIPT reporter compounds comprise a heterocyclic moiety comprising a basic nitrogen. In some embodiments, the oxygen atom of the ether (the hydroxyaryl oxygen atom) and the basic nitrogen are arranged in a 1,5-configuration relative to each other. In some embodiments, the heterocylic moiety is a five-membered ring.

Accordingly, in some embodiments, a silyl ether sensor compound of the present invention is represented by a compound of Formula (IA):

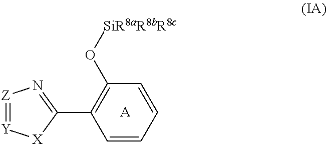

Wherein:
A is an optionally substituted aryl group;
X is NR, Y is CR³, and Z is CR⁴;
X is S, Y is CR³, and Z is CR⁴;
X is O, Y is CR³, and Z is CR⁴;
X is NR, Y is CR³, and Z is NR;
X is O, Y is CR³, and Z is N;
R is H, optionally substituted aryl or optionally substituted alkyl;
R³ is H, optionally substituted aryl or optionally substituted alkyl;
R⁴ is H, optionally substituted aryl or optionally substituted alkyl;
or R³ and R⁴, together with the carbon atoms to which they are attached, form a
cyclic moiety, for example an optionally substituted aryl moiety; and
each of R^{8a}, R^{8b}, R^{8c}, which may be the same or different, is C₁₋₆ alkyl or phenyl.

In some embodiments, X is NR, Y is CR³, and Z is CR⁴, thus forming an imidazole ring. In some embodiments, X is S, Y is CR³, and Z is CR⁴, thus forming a thiazole ring.

In some embodiments, a silyl ether sensor compound of the present invention is represented by a compound of Formula (IB):

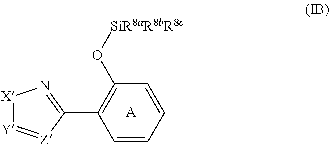

Wherein:
X′ is O, Y′ is CR³ and Z′ is CR⁴;
X′ is S, Y′ is CR³ and Z′ is CR⁴; or
X′ is NR, Y′ is CR³ and Z′ is CR⁴;
and A, R, R³, R⁴, R^{8a}, R^{8b}, and R^{8c} are as defined above for Formula (IA).

In some embodiments, the A moiety is an optionally substituted phenyl ring, or an optionally substituted fused aryl system such as naphthyl, anthracenyl, or phenanthryl. In some embodiments, the A moiety is a phenyl ring which may be optionally substituted. In some embodiments, the phenyl ring is unsubstituted.

In some embodiments, R³ and R⁴ are both hydrogen. In some embodiments, R³ and R⁴ together with the carbon atoms to which they are attached form a cyclic moiety. The cyclic moiety can be a carbocyclic or a heterocyclic ring of 5 to 8 ring atoms, preferably 5 or 6 ring atoms. The ring may be heterocyclyl, heteroaryl or aryl. The ring may be substituted by one or more substituents, or may be fused to form a polycyclic moiety. In some embodiments, R³ and R⁴ form an optionally substituted phenanthryl moiety.

In some embodiments, for example when the heterocycle formed by N, X, Y and Z is a thiazole, R³ and R⁴ form a phenyl ring thus forming a benzothiazole, which may be optionally substituted.

In some embodiments, for example when the heterocycle formed by N, X, Y and Z is an imidazole, R³ and R⁴ form an optionally substituted phenanthryl moiety. In some embodiments, the phenanthryl moiety is substituted by one or two, preferably two, substituents.

In some embodiments, R^{8a}, R^{8b}, and R^{8c} are all the same. In some embodiments, R^{8a} and R^{8b} are the same and R^{8c} is different. In some embodiments, the —SiR^{8a}R^{8b}R^{8c} group is —SiMe₃, —SiEt₃, —Si′BuMe₂, —Si′Pr₃, or —Si′BuPh₂.

It will be appreciated that, in preferred embodiments, the sensor compounds of Formula (IA) and Formula (IB) are silyl ethers of the corresponding ESIPT reporter compounds of Formula (IIA) and (IIB):

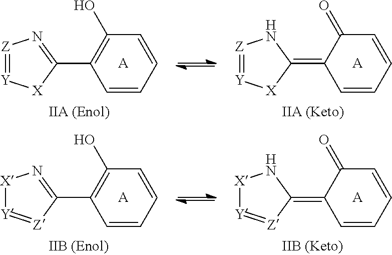

Wherein A, X, Y, Z, X′, Y′ and Z′ are as defined above for compounds of Formula (IA) and (IB).

It will be understood that the nature of the A ring and the X, Y and Z moieties (or X′, Y′ and Z′ moieties) will dictate the electronic properties of the chromophore and hence the wavelength(s) of the absorption of the chromophore of the compound of Formula (IIA) and (IIB). In preferred embodiments, the ESIPT reporter compound of Formula (IIA) or (IIB) has a chromophore with a main absorption having a wavelength of greater than or equal to 350 nm.

In some embodiments, the five-membered heterocycle formed by N, X, Y and Z in a compound of Formula (IA) is an imidazole, an oxadiazole, a thiazole, an oxazole or a 1,2,4-triazole. In some embodiments, the heterocycle is an imidazole. In some embodiments, the five-membered heterocycle formed by N, X′, Y′ and Z′ in a compound of Formula (IB) is an isoxazole, an isothiazole or a pyrazole.

It will be understood that, with respect to sensor compounds useful in the detection methods described herein, sensor compounds with structures and configurations other than those of compounds of Formulae (IA) and (IB) are also suitable.

In an embodiment, the sensor compound comprises a six-membered heterocyclic moiety comprising a basic nitrogen. Accordingly, there is also provided a sensor compound of Formula (IC):

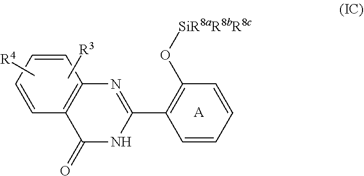

Wherein A, R³, R⁴, R^{8a}, R^{8b} and R^{8c} are as defined for compound (IA).

In an embodiment, the sensor compound is a compound of Formula (ID):

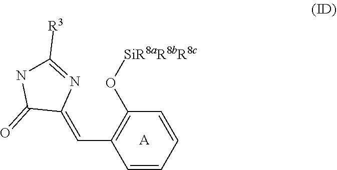

Wherein A, R³, R^{8a}, R^{8b} and R^{8c} are as defined for compound (IA).

It will be appreciated that a compound of Formula (ID) has a 1,6-configuration with respect to the position of the basic nitrogen atom and the phenol oxygen atom. As such, this embodiment could form a 7-membered transition state during keto-enol tautomerism.

In some embodiments of a compound of Formula (ID), the A ring is phenyl, optionally substituted with one or two substituents selected from alkyl, alkoxy, halo, cyano, nitro and haloalkyl.

In another embodiment, the sensor compound comprises an acyclic basic nitrogen atom in the form of an azine. Accordingly, in another embodiment, the sensor compound is a compound of Formula (IE):

Wherein:

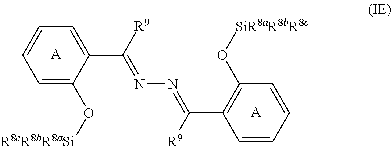

A, R^{8a}, R^{8b}, and R^{8c} are as defined for compound (IA); and
each R⁹ is hydrogen, optionally substituted alkyl or optionally substituted aryl.

In some embodiments of the sensor compound of Formula (IE), A is preferably phenyl optionally substituted by one or two substituents selected from alkyl, alkoxy and dialkylamino, for example C₁₋₆ alkyl, C₁₋₆alkylO- or (C₁₋₆alkyl)₂N—.

In another embodiment, the sensor compound comprises an acyclic basic nitrogen atom in the form of an imine. Accordingly, in another embodiment, the sensor compound is a compound of Formula (IF):

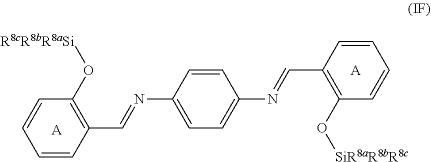

Wherein:
A, R^{8a}, R^{8b} and R^{8c} are as defined for compound (IA).

As used herein, the term “alkyl” is taken to include straight chain or branched chain monovalent saturated hydrocarbon groups, preferably having 1 to 20 carbon atoms, for example C₁₋₁₂, C₁₋₁₀, C₁₋₆ or C₁₋₄ alkyl. Examples of a straight chain alkyl group includes propyl, butyl, pentyl, hexyl, heptyl, octyl, dodecyl, and the like. Examples of a branched chain alkyl group includes iso-propyl, iso-butyl, sec-butyl, tert-butyl, iso-pentyl, neo-pentyl, and the like.

As used herein, the term “alkoxy group” is taken to include —O-alkyl groups, i.e. alkyl groups bound to an oxygen atom, preferably where the alkyl group has 1 to 20 carbon atoms, for example 1 to 4, or 1 to 6, or 1 to 8 carbon atoms. The alkoxy group may be straight chain or branched chain alkoxy groups. Examples of a straight chain alkoxy group includes propoxy, butoxy, pentoxy, hexyloxy, heptyloxy, octyloxy, dodecoxy and the like. Examples of a branched chain alkoxy group include (2-ethylhexyl)oxy. In some embodiments, an alkoxy group is a glycol based moiety, for example —O(CH₂CH₂O)CH₃.

As used herein, the term “aryl” refers to an unsaturated aromatic carbocyclic group having a single ring (e.g. phenyl) or multiple condensed rings (e.g. naphthyl, anthracenyl, phenanthryl), preferably having from 6 to 14 carbon atoms. Examples of aryl groups include phenyl, naphthyl, anthracenyl, phenanthryl, and the like. In some embodiments, aryl is preferably phenyl. Aryl groups may be optionally substituted. Where one or more carbon atoms of the aryl group is replaced with one or more heteroatoms, the group is a heteroaryl group. The heteroatoms may be selected from nitrogen, oxygen and sulphur. Examples of heteroaryl groups include furanyl, quinazolinyl, quinolinyl, isoquinolinyl, indolyl, isoindolyl, benzopyranyl, benzooxazolyl, benzimidazolyl, pyrazolyl, tetrazolyl, oxazolyl, oxadiazolyl, isoxazolyl, thiadiazolyl, quinolizinyl, pyranyl, isothiazolyl, thiazolyl, thienyl (thiophenyl), imidazolyl, pyrazinyl, pyridazinyl, pyrimidinyl, isothiazolyl, pyridyl, triazolyl, benzothienyl, pyrrolyl, benzothiazolyl, quinoxalinyl, naphthyridinyl, pteridinyl, carbazolyl, azepinyl, acridinyl, benzisothiazolyl, benzoxazolyl, benzisoxazolyl, benzofuryl, purinyl, benzimidazolyl, triazinyl, carbazolyl, and the like.

As used herein, the term “halogen” refers to a fluorine, chlorine, bromine or iodine group.

As used herein, the term “optionally substituted” as used throughout the specification denotes that the group may or may not be further substituted or fused (so as to form a condensed polycyclic system), with one or more non-hydrogen substituent groups. In certain embodiments the substituent groups are one or more groups independently selected from the group consisting of halogen, ═O, ═S, —CN, —NO₂, —CF₃, —OCF₃, alkyl, alkenyl, alkynyl, alkoxy, haloalkyl, haloalkenyl, haloalkynyl, haloalkoxy, heteroalkyl, cycloalkyl, cycloalkenyl, heterocycloalkyl, heterocycloalkenyl, aryl, heteroaryl, cycloalkylalkyl, heterocycloalkylalkyl, heteroarylalkyl, arylalkyl, cycloalkylalkenyl, heterocycloalkylalkenyl, arylalkenyl, heteroarylalkenyl, cycloalkylheteroalkyl, heterocycloalkylheteroalkyl, arylheteroalkyl, heteroarylheteroalkyl, hydroxy, hydroxyalkyl, alkyloxy, alkyloxyalkyl, alkyloxycycloalkyl, alkyloxyheterocycloalkyl, alkyloxyaryl, alkyloxyheteroaryl, alkyloxycarbonyl, alkylaminocarbonyl, alkenyloxy, alkynyloxy, cycloalkyloxy, cycloalkenyloxy, heterocycloalkyloxy, heterocycloalkenyloxy, aryloxy, phenoxy, benzyloxy, heteroaryloxy, arylalkyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, alkylsulfinyl, arylsulfinyl, aminosulfinylaminoalkyl and carbonyl.

In some embodiments, a substituent may be a glycol moiety such as an ethylene glycol chain, for example 2-methoxymethyl, 2-methoxyethyl, 2-(2-methoxyethoxy)ethyl, and 2-(2-(2-methoxyethoxy)ethoxy)ethyl. In some embodiments, a substituent may be —O(CH₂CH₂O)ₙC₁₋₄alkyl, wherein n is 1-10. In some embodiments, a substituent can be a dendron that include one or more aryl rings (preferably phenyl).

In some embodiments each optional substituent is independently selected from the group consisting of: halogen, ═O, ═S, —CN, —NO₂, —CF₃, —OCF₃, alkyl, alkenyl, alkynyl, haloalkyl, haloalkenyl, haloalkynyl, heteroalkyl, cycloalkyl, cycloalkenyl, heterocycloalkyl, heterocycloalkenyl, aryl, heteroaryl, hydroxy, hydroxyalkyl, alkyloxy, alkyloxyalkyl, alkyloxyaryl, alkyloxyheteroaryl, alkenyloxy, alkynyloxy, cycloalkyloxy, cycloalkenyloxy, heterocycloalkyloxy, heterocycloalkenyloxy, aryloxy, heteroaryloxy, arylalkyl, heteroarylalkyl, arylalkyloxy, amino, alkylamino, acylamino, aminoalkyl, arylamino, sulfonyl, alkylsulfonyl, arylsulfonyl, aminosulfonyl, aminoalkyl, —COOH, —SH, and acyl.

In some embodiments, optional substituents for aryl groups include halogen, e.g. —F, —Cl or —Br; —CN; haloalkyl, e.g. —CF₃; haloalkoxy, e.g. —OCF₃; amino, e.g. —N(C₁₋₆ alkyl)₂; C₁₋₆ alkyl; —C₁₋₆ alkoxy, e.g. —OCH₃ or (2-ethylhexyl)oxy; and optionally substituted phenyl. In some embodiments, a substituent is —O(CH₂CH₂O)CH₃. In some embodiments, a substituent is 4-((2-ethylhexyl)oxy)phenyl-. In some embodiments, an optional substituent for an aryl group is —O(CH₂CH₂O)ₙalkyl, wherein n is 2-20. In some embodiments, optionally substituted phenyl includes bis(4-((2-ethylhexyl)oxy) phenyl)-.

In some embodiments, an aryl substituent is a silyl ether group —OSiR^{x}R^{y}R^{z} wherein R^{x}, R^{y} and R^{z} are each selected from C₁₋₆alkyl and phenyl. In this embodiment, the sensor compound has two or more silyl ether groups. In some embodiments, when the sensor compound has two silyl ether groups it is preferably symmetrical.

In some embodiments, the silyl ether substituent is —OSiR^{8a}R^{8b}R^{8c} wherein R^{8a}, R^{8b}, and R^{8c} are as defined above for compounds of Formula (IA).

In some embodiments, the compound of Formula (IA) is a compound of Formula (Ia):

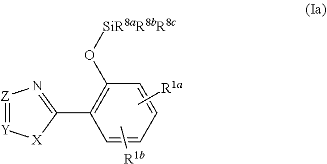

Wherein:
R^{1a} and R^{1b} are each selected from hydrogen, optionally substituted alkyl and optionally substituted aryl; and
X, Y, Z, R^{8a}, R^{8b} and R^{8c} are as defined above for compounds of Formula (IA).

In some embodiments, the compound of Formula (IA) or (Ia) is a compound of Formula (Ib):

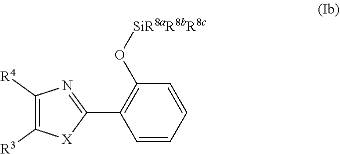

Wherein:
X is S or NR;
R is hydrogen, C₁₋₁₀ alkyl or optionally substituted phenyl;
R³ is hydrogen, optionally substituted aryl or optionally substituted alkyl;
R⁴ is hydrogen, optionally substituted aryl or optionally substituted alkyl;
or R³ and R⁴ together with the carbon atoms to which they are attached form a cyclic moiety, for example an optionally substituted aryl group; and
R^{8a}, R^{8b}, R^{8c} are each independently selected from C₁₋₆ alkyl or phenyl.

In some embodiments, the compound of Formula (IA), (Ia) or (Ib) is a compound of Formula (Ic):

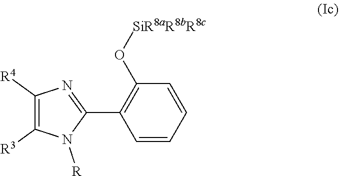

Wherein R, R³, R⁴, R^{8a}, R^{8b} and R^{8c} are as defined above.

In some embodiments, the compound of Formula (I), (Ia), (Ib) or (Ic) is a compound of Formula (Id):

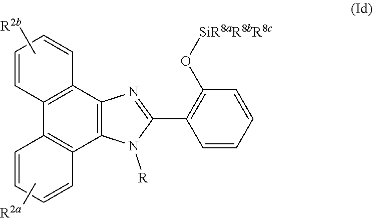

Wherein:
R is C₁₋₁₀ alkyl; or optionally substituted phenyl;
each R^{2a} and R^{2b}, which may be the same or different, is hydrogen, optionally substituted phenyl or C₁₋₁₀ alkyl; and
each of R^{8a}, R^{8b} and R^{8c}, which may be the same or different is C₁₋₄ alkyl or phenyl.

Optional substituents for aryl groups include one or two substituents selected from C₁₋₄ alkyl, for example n-butyl; C₁₋₂haloalkyl, for example trifluoromethyl; C₁₋₁₀alkoxyphenyl, for example 4-alkoxyphenyl such as 4-((2-ethylhexyl)oxy) phenyl; and —O(CH₂CH₂O)ₙC₁₋₄alkyl wherein n is 2-6, for example —O(CH₂CH₂O)₂CH₃.

In an embodiment, a substituted alky group is 3,5-bis[4-((2-ethylhexyl)oxy)phenyl]phenyl.

In some embodiments, R is hydrogen, C₁₋₄ alkyl; or phenyl optionally substituted with C₁₋₆alkyl, C₁₋₄haloalkyl or O(CH₂CH₂O)ₙC₁₋₄alkyl. In an embodiment, R is hydrogen. In an embodiment, R is —phenyl-O(CH₂CH₂O)ₙC₁₋₄alkyl.

In some embodiments, the compound of Formula (I) is a benzothiazole compound of Formula (Ie):

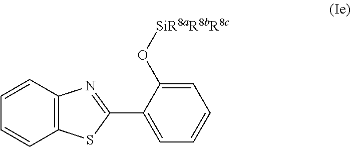

wherein each of R^{8a}, R^{8b} and R^{8c}, which may be the same or different is C₁₋₄ alkyl or phenyl.

Specific examples of sensor compounds of the invention are shown below. Compounds are referred to by reference numbers of the form “SQF XXXX” for convenience. In some embodiments, the compound of Formula (IA) is an imidazole derivative, for example:

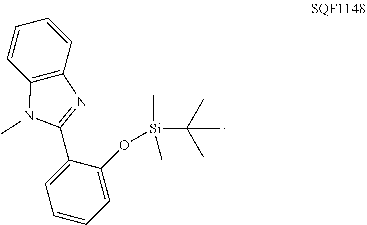 [SMILES: Cn1c(-c2ccccc2O[Si](C)(C)C(C)(C)C)nc2ccccc21]

In some embodiments, the compound of Formula (IA) is:

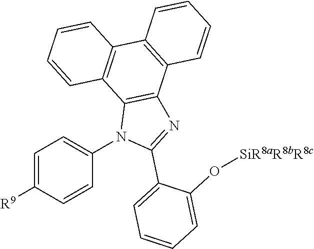

| Compound | R^{8a}R^{8b}R^{8c} | R⁹ |
|---|---|---|
| SQF1323 | CH₃, CH₃, tert-C₄H₉ | n-C₄H₉ |
| SQF1360 | CH₃, CH₃, tert-C₄H₉ | CF₃ |
| SQF1370 | CH₃, CH₃, tert-C₄H₉ | H |
| SQF1382 | CH₃, CH₃, tert-C₄H₉ | CH₃ |
| SQF1388 | iso-C₃H₇, iso-C₃H₇, iso-C₃H₇ | H |
| SQF1389 | Ph, Ph, tert-C₄H₉ | H |
| SQF1399 | CH₃, CH₃, CH₃ | H |
| SQF13100 | C₂H₅, C₂H₅, C₂H₅ | H |
| SQF13111 | CH₃, CH₃, tert-C₄H₉ | O(CH₂CH₂O)₂CH₃ |

In some embodiments, the compound of Formula (IA) is:

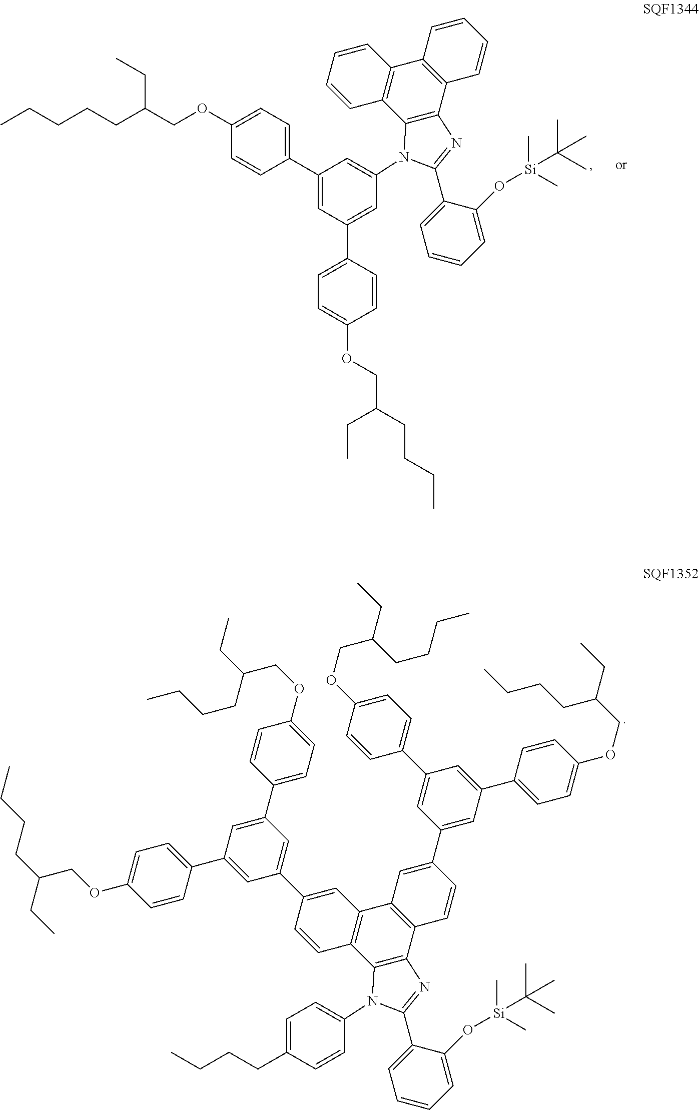 [SMILES: CCCCCC(CC)COc1ccc(-c2cc(-c3ccc(OCC(CC)CCCC)cc3)cc(-n3c(-c4ccccc4O[Si](C)(C)C(C)(C)C)nc4c5ccccc5c5ccccc5c43)c2)cc1 ; CCCCc1ccc(-n2c(-c3ccccc3O[Si](C)(C)C(C)(C)C)nc3c4ccc(-c5cc(-c6ccc(OCC(CC)CCCC)cc6)cc(-c6ccc(OCC(CC)CCCC)cc6)c5)cc4c4cc(-c5cc(-c6ccc(OCC(CC)CCCC)cc6)cc(-c6ccc(OCC(CC)CCCC)cc6)c5)ccc4c32)cc1]

In some embodiments, the compound of Formula (IA) is a thiazole derivative:

| 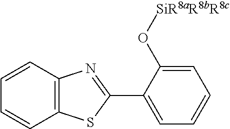 | |
|---|---|
| Compound | R^{8a}R^{8b}R^{8c} |
| SQF1140 | CH₃, CH₃, tert-C₄H₉ |
| SQF1393 | CH₃, CH₃, CH₃ |
| SQF1394 | C₂H₅, C₂H₅, C₂H₅ |
| SQF1395 | iso-C₃H₇, iso-C₃H₇, iso-C₃H₇ |
| SQF1396 | Ph, Ph, tert-C₄H₉ |

It will be understood that definitions given to the variables of the generic formulae described herein will result in molecular structures that are in agreement with standard organic chemistry definitions and atom valencies.

The compound of the invention may be dendritic in character, for example having a “core” comprising one or more of the sensor compounds with dendron moieties attached to the core. Alternatively, the dendron moieties may comprise sensor compounds. The dendrons can be first, second or higher generations, with surface groups chosen to provide the necessary solubility and interactions with the analyte.

The sensor compound may be in the form of a polymer. In this embodiment, the sensor compound includes multiple moieties of the sensor compound.

Polymeric materials comprising sensor compounds in accordance with the invention may be formed from multiple chromophore units linked by linker groups to form a polymer backbone. Alternatively, the sensor compounds may be attached to a polymer backbone to form a plurality of pendant functional groups or side chains. It will be understood that the polymeric sensor compound may be in the form of, for example, a co-polymer, a homopolymer or a functionalized polymer, for example where the chromophore units are attached to form side chains on a polymer backbone.

In some preferred embodiments, the sensor compound is non-polymeric. In some embodiments, the sensor compound is not dendritic.

In some embodiments, the sensor compounds useful in the invention are small molecules. Typically, a small molecule sensor compound will have a molecular weight of less than 2000, but if the compounds have a dendritic architecture the molecular weight could be higher. Small molecules and dendrimers may offer an advantage of providing reproducibility or consistency over polymeric sensor compounds.

Polymeric sensor compounds are less amenable to reproducible synthesis than small molecules or dendrimers. Typically, batch-to-batch variations during polymer synthesis may result in less reliable or less reproducible sensing properties between batches leading to inconsistent performance between batches.

In accordance with the present invention it will be understood that there is provided novel fluorescent sensor compounds. Advantageously, these materials find application in detection of a nerve agent comprising a P—F bond such as a G-series nerve agent such as Sarin, Cyclosarin and Soman or another nerve agent having a P—F bond. In some embodiments, the compounds of the present invention are selective for detection of HF present in a G-series nerve agent. In some embodiments, the materials provide a rapid reaction and provide access to real time detection of nerve agents. In some examples, the compounds have been found to demonstrate selectivity. Accordingly, the present invention further provides a sensor compound of the Formula (IA), (IB), (IC), (ID), (IE), or (IF) as hereinbefore defined. The compounds of Formula (IA), (IB), (IC), (ID), (IE), or (IF) may, collectively be referred to as compounds of Formula (I).

In some preferred embodiments, the compound is a compound of Formula (Id) or a compound of Formula (Ie).

In some embodiments, the compound of Formula (I) is selected from SQF1148, SQF1323, SQF1360, SQF1370, SQF1382, SQF1388, SQF1389, SQF1399, SQF13100, SQF13111, SQF1344, SQF1352, SQF1140, SQF1393, SQF1394, SQF1395, and SQF1396.

Methods of Synthesis

The silyl ether sensor compounds as described herein may be synthesized from commercially available starting materials using recognized multi-step synthetic routes known in the art. The preparation of specific compounds is described in the Examples below. It will be appreciated that these routes and methodologies can be adapted to synthesize other sensor compounds of the invention.

The sensor compounds of the invention are silyl phenol ethers. Methods for preparing silyl ethers are well known in the art. In general, silyl ethers may be prepared by reacting the desired hydroxy compound with the desired silyl triflate and a hindered amine base. Alternatively, the hydroxyl compound can be reacted with the appropriate silyl chloride in the presence of imidazole and a solvent such as NN-dimethylformamide or dichloromethane.

In some preferred embodiments, the silyl ethers of the invention can be prepared by reacting the appropriate phenol compound with the desired substituted silyl chloride in the presence of imidazole and a solvent, for example, dimethylformamide, in an inert atmosphere as shown in the exemplary scheme below.

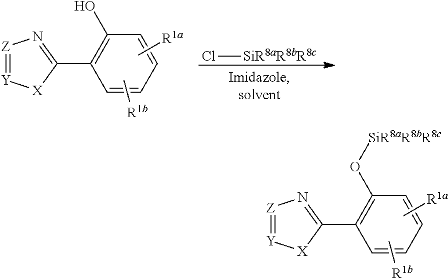

Phenol compounds useful for preparation of the sensor compounds of Formula (IA) and (IB) can be prepared in accordance with known methods for the preparation of phenols. Exemplary methods are described in the Examples below. It will be understood that other phenol compounds may be prepared using analogous routes to those described in the examples.

In some embodiments, phenol precursors of the sensor compounds (IA) and (IB) can be prepared using routes analogous to those described in, for example, K. Wang, F. Zhao, C. Wang, S. Chen, D. Chen, H. Zhang, Y. Liu, D. Ma, Y. Wang, Adv. Funct. Mater., 2013, 23, 2672-2680; T. Shida, T. Mutai, K. Araki, CrystEngComm, 2013, 15, 10179-10182; and F. S. Santos, E. Ramasamy, V. Ramamurthy & F. S. Rodembusch,. J. Mater. Chem. C, 2016, 4, 2820-2827.

Sensor compounds of Formula (IC) can be prepared through silylation of the corresponding phenol compound. The phenol starting materials may be commercially available, or may be made in accordance with S. P. Anthony, Chem.—Asian J., 2012, 7, 374-379.

Sensor compounds of Formula (ID), wherein the basic nitrogen atom is located in a 1,6-configuration relative to the position of the keto-enol oxygen atom can be prepared through silylation of the corresponding phenol compound. The phenol precursor can be made in accordance with the methodology of, for example, J. Org. Chem. 2011, 76, 20, 8189-8202.

An azine phenol precursor of the silyl ether sensor compound of Formula (IE) may be prepared in accordance with the methodology described in J. Phys. Chem. C 2013, 117, 3467-3474.

An acyclic imine phenol precursor of the silyl ether sensor compound of Formula (IF) may be prepared in accordance with the methods described in Langmuir 2014, 30, 9, 2351-2359.

One skilled in the art would understand that a wide variety of substitution patterns around the sensor compound of Formula (I) can be accessed by judicious choice of starting materials, reagents and appropriate reaction conditions.

Certain substituents in any of the reaction intermediates or compounds of Formula (I) may be converted to other substituents by conventional methods known to those skilled in the art. For example a substituent R¹ may be converted to another substituent R¹; or a substituent R² may be converted to a different R² substituent. Such transformations are well known in the art and are described in, for example, Richard Larock, Comprehensive Organic Transformations, 2^{nd} Edition, Wiley, ISBN 0-417-19031-4.

It will be appreciated that it may be necessary to protect certain substituents during one or more of the above procedures. Those skilled in the art will recognize when a protecting group is required. Standard protection and deprotection techniques, such as those described in Peter G. M. Wuts, Greene's Protective Groups in Organic Synthesis, Wiley, New York, 2014 ISBN 9781118057483, may be used. It will be appreciated that protecting groups may be interconverted using conventional means.

The reactions and processes described herein may employ conventional laboratory techniques for heating and cooling, such as thermostatically controlled oil baths or heating blocks and ice baths or solid CO₂/acetone baths. Use of inert atmospheric conditions such as nitrogen or argon may be employed. Conventional methods of isolation of the desired compound, such as extraction or precipitation techniques, and the like, may be used. Organic solvents or solutions may be dried where required using standard, well-known techniques. Purification of compounds or intermediates may be effected using conventional techniques such as chromatography and/or crystallisation.

In order that the invention may be readily understood and put into practical effect, particular preferred embodiments will now be described by way of the following non-limiting examples.

### Examples

Materials synthesis: All reagents were purchased from commercial sources and were used as received unless otherwise stated. Solvents for chromatography were distilled prior to use. Column chromatography was performed using Davisil LC60A micron silica gel, Merck Aluminium Oxide 90 active neutral (70-239 mesh), or Bio-Beads S—X1 Support. ¹H and ¹³C NMR were performed using Bruker Avance 500 MHz spectrometers in deuterated dichloromethane referenced to 5.32 ppm for ¹H and 53.8 ppm for ¹³C; in deuterated chloroform referenced to 7.26 ppm for ¹H and 77.0 ppm for ¹³C. Coupling constants are given to the nearest 0.5 Hz. UV-visible spectrophotometry was performed using either a Cary 5000 UV-Vis spectrophotometer in dichloromethane or ethanol solution, or OceanOptics Flame spectrometer on thin films on quartz substrates, with absorbance shoulders denoted as sh. FT-IR spectroscopy was performed on solid samples using a Perkin-Elmer Spectrum 100 FT-IR spectrometer with an ATR attachment. Melting points (MPs) were measured in a glass capillary on a Büchi B-545 melting point apparatus and are uncorrected. Microanalyses were performed using a Carlo Erba NA 1500 Elemental Analyzer. High resolution electrospray ionisation (HRMS) accurate mass measurements were recorded in positive mode on a Bruker MicroTOF-Q (quadrupole-time of flight) instrument with a Bruker ESI source.

Chemical warfare nerve agent simulants such as DFP are available from commercial sources.

### Preparation of SQF1399

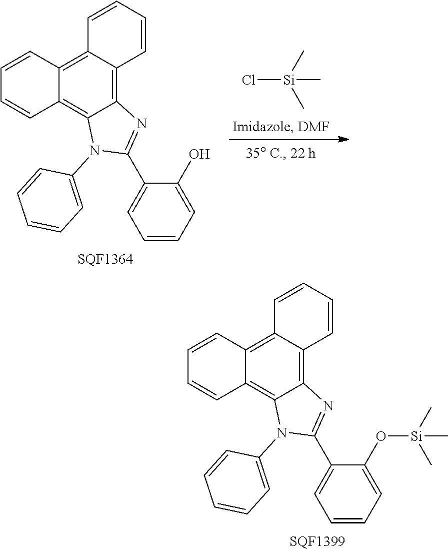

A mixture of SQF1364(K. Wang, F. Zhao, C. Wang, S. Chen, D. Chen, H. Zhang, Y. Liu, D. Ma, Y. Wang, Adv. Funct. Mater., 2013, 23, 2672-2680.) (580 mg, 1.5 mmol), trimethylsilyl chloride (0.30 mL, 2.3 mmol), imidazole (337 mg, 5.0 mmol), and anhydrous dimethylformamide (10 mL) was stirred under argon in an oil bath held at 35° C. for 22 h. The mixture was allowed to cool to room temperature and then water (50 mL) and ethylacetate (50 mL) were added. The organic phase was separated and washed with water (5×50 mL), dried over anhydrous magnesium sulfate, and filtered. The filtrate was collected and the solvent removed. The residue was purified by column chromatography over Bio-Beads S—X1 support using toluene as eluent to give a white solid (460 mg, 67%). mp 183-184° C. λₘₐₓ(dichloromethane)/nm: 260 (log ε/dm³ mol⁻¹ cm⁻¹ 4.82), 284 (4.27), 295 sh (4.11), 308 (4.05), 342 (3.39), 358 (3.43).) λₘₐₓ(fluorescence) (dichloromethane)/nm: 367, 375, 403 sh. ¹H NMR (δ, 500 MHz, CD₂Cl₂): 0.09 (6H, s, —CH₃), 6.77 (1H, dd, J=1.0, J=8.0, O-Phenyl-H), 6.98 (1H, ddd, J=1.0, J=7.5, J=7.5, O-Phenyl-H), 7.25-7.30 (3H, m, Phenanthro-H and O-Phenyl-H), 7.41-7.51 (6H, m, N-Phenyl-H and O-Phenyl-H), 7.52 (1H, ddd, J=2.0, J=6.5, J=8.5, Phenanthro-H), 7.66 (1H, ddd, J=1.0, J=7.0, J=8.5, Phenanthro-H), 7.73 (1H, ddd, J=1.0, J=7.0, J=8.0, Phenanthro-H), 8.73-8.76 (2H, m, Phenanthro-H), 8.79-8.80 (1H, m, Phenanthro-H). ¹³C NMR (δ, 125 MHz, CD₂Cl₂): 0.31, 120.0, 121.3, 121.4, 122.8, 123.5, 123.9, 124.3, 125.1, 125.6, 126.6, 127.5(7), 127.6(1), 127.9, 128.4, 129.0, 129.3, 129.4, 129.5, 131.1, 132.7, 137.6, 138.5, 150.4, 154.6. m/z [HRMS-ESI⁺]: expected 459.1887 ([M+H^{]}+), found: 459.1895 ([M+H]⁺).

### Preparation of SQF13100

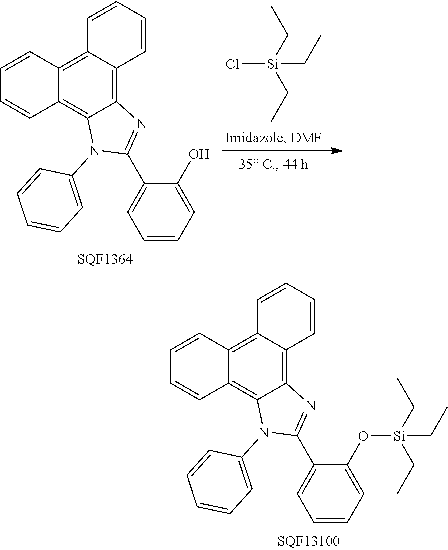

A mixture of SQF1364 (K. Wang, F. Zhao, C. Wang, S. Chen, D. Chen, H. Zhang, Y. Liu, D. Ma, Y. Wang, Adv. Funct. Mater., 2013, 23, 2672-2680.) (580 mg, 1.5 mmol), triethylsilyl chloride (0.40 mL, 2.3 mmol), imidazole (337 mg, 5.0 mmol), and anhydrous dimethylformamide (10 mL) was stirred under argon in an oil bath held at 35° C. for 44 h. The mixture was allowed to cool to room temperature and then water (50 mL) and ethylacetate (50 mL) were added. The organic phase was separated and washed with water (5×50 mL), dried over anhydrous magnesium sulfate, and filtered. The filtrate was collected and the solvent removed. The residue was purified by column chromatography over Bio-Beads S—X1 support using toluene as eluent to give a white solid (447 mg, 60%). mp 111-112° C. λₘₐₓ(dichloromethane)/nm: 260 (log ε/dm³ mol⁻¹ cm⁻¹ 4.87), 284 (4.31), 295 sh (4.14), 308 (4.09), 342 (3.39), 358 (3.46). λₘₐₓ(fluorescence) (dichloromethane)/nm: 366, 374, 401 sh. ¹H NMR (δ, 500 MHz, CDCl₃): 0.61 (6H, q, J=8.0, —CH₂—), 0.77 (9H, t, J=8.0, —CH₃), 6.73 (1H, dd, J=0.5, J=8.0, O-Phenyl-H), 6.92 (1H, ddd, J=1.0, J=7.5, J=8.5, O-Phenyl-H), 7.19-7.29 (3H, m, Phenanthro-H and O-Phenyl-H), 7.39 (1H, dd, J=2.0, J=7.5, O-Phenyl-H), 7.41-7.47 (5H, m, N-Phenyl-H), 7.51 (1H, ddd, J=2.0, J=6.5, J=8.5, Phenanthro-H), 7.64 (1H, ddd, J=1.5, J=7.0, J=8.5, Phenanthro-H), 7.72 (1H, ddd, J=1.0, J=7.0, J=8.0, Phenanthro-H), 8.72 (1H, d, J=8.5, Phenanthro-H), 8.79 (1H, d, J=8.5, Phenanthro-H), 8.83-8.84 (1H, m, Phenanthro-H). ¹³C NMR (δ, 125 MHz, CDCl₃): 5.0, 6.4, 119.1, 120.7, 120.9, 122.7, 123.0(1), 123.0(5), 124.0, 124.6, 125.2, 126.1, 127.1, 127.5, 128.1, 128.5, 129.0(0), 129.0(4), 129.1, 130.7, 132.3, 137.3, 138.1, 150.1, 154.5. m/z [HRMS-ESI⁺]: expected 501.2357 ([M+H]⁺), found: 501.2372 ([M+H]⁺).

### Preparation of SQF1370

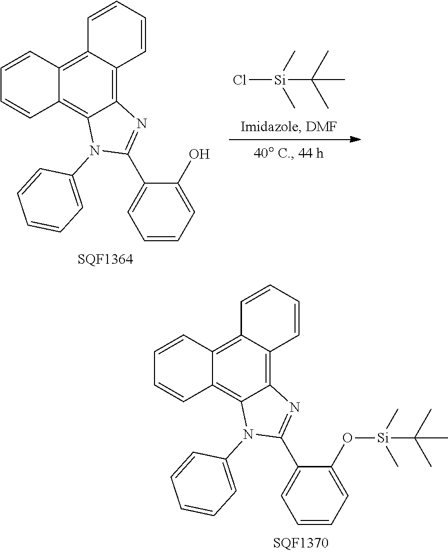

A mixture of SQF1364 (K. Wang, F. Zhao, C. Wang, S. Chen, D. Chen, H. Zhang, Y. Liu, D. Ma, Y. Wang, Adv. Funct. Mater., 2013, 23, 2672-2680.) (773 mg, 2.0 mmol), tert-butyldimethylsilyl chloride (452 mg, 3.0 mmol), imidazole (450 mg, 6.6 mmol), and anhydrous dimethylformamide (10 mL) was stirred under argon in an oil bath held at 40° C. for 44 h. The mixture was allowed to cool to room temperature and then water (50 mL) and ethylacetate (50 mL) were added. The organic phase was separated and washed with water (5×50 mL), dried over anhydrous magnesium sulfate, and filtered. The filtrate was collected and the solvent removed. The residue was purified by column chromatography over silica using dichloromethane:hexane (1:1) and then dichloromethane:ethylacetate (1:0 to 4:1) as eluent. The product was further purified by recrystallization using dichloromethane and methanol to give a white solid (772 mg, 77%). mp 169-170° C. λₘₐₓ(dichloromethane)/nm: 259 (log ε/dm³ mol⁻¹ cm⁻¹ 4.86), 284 (4.29), 295 sh (4.13), 308 (4.06), 340 (3.37), 357 (3.43). λₘₐₓ(fluorescence) (dichloromethane)/nm: 365, 374, 400 sh. ¹H NMR (δ, 500 MHz, CD₂Cl₂): 0.10 (6H, s, —CH₃), 0.71 (9H, s, —C(CH₃)₃), 6.83 (1H, dd, J=1.0, J=8.5, O-Phenyl-H), 6.93 (1H, ddd, J=1.0, J=7.5, J=7.5, O-Phenyl-H), 7.22 (1H, ddd, J=0.5, J=1.5, J=8.5, Phenanthro-H), 7.25-7.29 (2H, m, Phenanthro-H and O-Phenyl-H), 7.32 (1H, dd, J=2.0, J=7.5, O-Phenyl-H), 7.45-7.49 (5H, m, N-Phenyl-H), 7.52 (1H, ddd, J=1.5, J=7.0, J=8.5, Phenanthro-H), 7.65 (1H, ddd, J=1.5, J=7.0, J=8.5, Phenanthro-H), 7.72 (1H, ddd, J=1.0, J=7.0, J=8.0, Phenanthro-H), 8.73-8.76 (2H, m, Phenanthro-H), 8.79 (1H, ddd, J=0.5, J=0.5, J=8.5, Phenanthro-H). ¹³C NMR (δ, 125 MHz, CD₂Cl₂): −4.4, 18.2, 25.5, 120.1, 121.1, 121.3, 122.9, 123.4, 123.5, 123.9, 124.3, 125.1, 125.6, 126.6, 127.5, 127.7, 127.9, 128.4, 128.9, 129.3, 129.5, 129.7, 131.1, 132.4, 137.5, 138.5, 150.1, 155.2. m/z [HRMS-ESI⁺]: expected 501.2357 ([M+H]⁺), found: 501.2355 ([M+H]⁺).

### Preparation of SQF1388

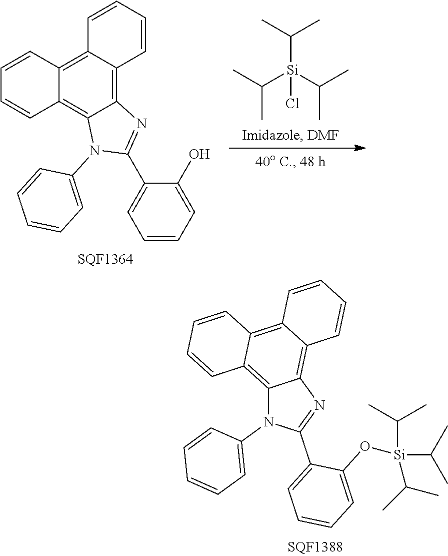 [SMILES: Oc1ccccc1-c1nc2c3ccccc3c3ccccc3c2n1-c1ccccc1 ; CC(C)[Si](Cl)(C(C)C)C(C)C ; CC(C)[Si](Oc1ccccc1-c1nc2c3ccccc3c3ccccc3c2n1-c1ccccc1)(C(C)C)C(C)C]

A mixture of SQF1364 (K. Wang, F. Zhao, C. Wang, S. Chen, D. Chen, H. Zhang, Y. Liu, D. Ma, Y. Wang, Adv. Funct. Mater., 2013, 23, 2672-2680.) (580 mg, 1.5 mmol), triisopropylsilyl chloride (0.50 mL, 2.3 mmol), imidazole (337 mg, 5.0 mmol), and anhydrous dimethylformamide (10 mL) was stirred under argon in an oil bath held at 40° C. for 48 h. The mixture was allowed to cool to room temperature and then water (50 mL) and ethylacetate (50 mL) were added. The organic phase was separated and washed with water (5×50 mL), dried over anhydrous magnesium sulfate, and filtered. The filtrate was collected and the solvent removed. The residue was purified by recrystallization using dichloromethane and methanol to give a white solid (534 mg, 65%). mp 179-180° C. λₘₐₓ(dichloromethane)/nm: 259 (log ε/dm³ mol⁻¹ cm⁻¹ 4.86), 284 (4.31), 295 sh (4.13), 308 (4.07), 340 (3.41), 358 (3.47). λₘₐₓ(fluorescence) (dichloromethane)/nm: 365, 374, 401 sh. ¹H NMR (δ, 500 MHz, CDCl₃): 0.92 (18H, d, J=7.5, —CH₃), 1.19 (3H, septet, J=7.5, —CH), 6.79 (1H, dd, J=1.0, J=8.5, O-Phenyl-H), 6.86 (1H, ddd, J=1.0, J=7.5, J=8.5, O-Phenyl-H), 7.19 (1H, ddd, J=2.0, J=7.5, J=8.5, O-Phenyl-H), 7.23 (1H, ddd, J=0.5, J=1.5, J=8.5, Phenanthro-H), 7.27 (1H, ddd, J=1.0, J=7.0, J=8.5, Phenanthro-H), 7.30 (1H, dd, J=1.5, J=7.5, O-Phenyl-H), 7.39-7.47 (5H, m, N-Phenyl-H), 7.51 (1H, ddd, J=1.5, J=7.0, J=8.5, Phenanthro-H), 7.64 (1H, ddd, J=1.5, J=7.0, J=8.5, Phenanthro-H), 7.72 (1H, ddd, J=1.0, J=7.0, J=8.0, Phenanthro-H), 8.71-8.73 (1H, m, Phenanthro-H), 8.77-8.79 (1H, m, Phenanthro-H), 8.81 (1H, dd, J=1.0, J=7.5, Phenanthro-H). ¹³C NMR (δ, 125 MHz, CDCl₃): 12.6, 17.7, 118.7, 120.2, 120.9, 122.6, 122.7, 122.9, 123.0, 124.0, 124.6, 125.1, 126.0, 127.0, 127.1, 127.5, 128.0, 128.4, 129.0(0), 129.0(1), 129.2, 130.6, 132.2, 137.2, 138.1, 150.0, 155.0. m/z [HRMS-ESI⁺]: expected 543.2826 ([M+H]⁺), found: 543.2842 ([M+H]⁺).

### Preparation of SQF1389

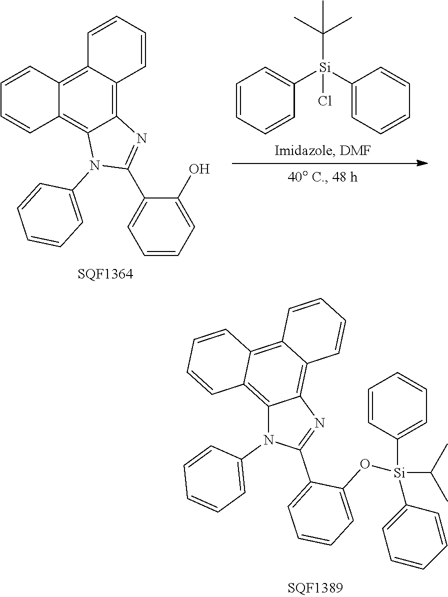 [SMILES: Oc1ccccc1-c1nc2c3ccccc3c3ccccc3c2n1-c1ccccc1 ; CC(C)(C)[Si](Cl)(c1ccccc1)c1ccccc1 ; CC(C)[Si](Oc1ccccc1-c1nc2c3ccccc3c3ccccc3c2n1-c1ccccc1)(c1ccccc1)c1ccccc1]

A mixture of SQF1364 (K. Wang, F. Zhao, C. Wang, S. Chen, D. Chen, H. Zhang, Y. Liu, D. Ma, Y. Wang, Adv. Funct. Mater., 2013, 23, 2672-2680.) (580 mg, 1.5 mmol), tert-butyldiphenylsilyl chloride (0.58, 2.3 mmol), imidazole (337 mg, 5.0 mmol), and anhydrous dimethylformamide (10 mL) was stirred under argon in an oil bath held at 40° C. for 48 h. The mixture was allowed to cool to room temperature and then water (50 mL) and ethylacetate (50 mL) were added. The organic phase was separated and washed with water (5×50 mL), dried over anhydrous magnesium sulfate, and filtered. The filtrate was collected and the solvent removed. The residue was purified by column chromatography over silica using dichloromethane:hexane (1:1) and then dichloromethane:ethylacetate (4:1) as eluent to give a white solid (541 mg, 57%). mp 217-218° C. λₘₐₓ(dichloromethane)/nm: 259 (log ε/dm³ mol⁻¹ cm⁻¹ 4.87), 284 (4.31), 295 sh (4.13), 308 (4.07), 339 (3.36), 357 (3.44). λₘₐₓ(fluorescence) (dichloromethane)/nm: 364, 373, 401 sh. ¹H NMR (δ, 500 MHz, CDCl₃): 0.82 (9H, s, —CH₃), 6.43 (1H, ddd, J=0.5, J=1.0, J=8.0, O-Phenyl-H), 6.82 (1H, ddd, J=1.0, J=7.5, J=8.5, O-Phenyl-H), 6.93 (1H, ddd, J=2.0, J=7.5, J=8.5, O-Phenyl-H), 7.20-7.23 (5H, m, Si-Phenyl-H and Phenanthro-H), 7.26 (1H, ddd, J=1.0, J=7.0, J=8.5, Phenanthro-H), 7.32-7.37 (3H, m, Si-Phenyl-H and O-Phenyl-H), 7.39-7.42 (4H, m, N-Phenyl-H), 7.45-7.49 (1H, m, N-Phenyl-H), 7.52 (1H, ddd, J=1.5, J=7.0, J=8.5, Phenanthro-H), 7.55-7.57 (4H, m, Si-Phenyl-H), 7.66 (1H, ddd, J=1.5, J=7.0, J=8.5, Phenanthro-H), 7.73 (1H, ddd, J=1.0, J=7.0, J=8.0, Phenanthro-H), 8.74-8.76 (1H, m, Phenanthro-H), 8.80-8.82 (1H, m, Phenanthro-H), 8.87-8.89 (1H, m, Phenanthro-H). ¹³CNMR (δ, 125 MHz, CDCl₃): 19.4, 26.3, 119.2, 120.4, 120.9, 122.4, 122.8, 123.0(1), 123.0(4), 124.0, 124.7, 125.3, 126.1, 127.2, 127.3, 127.5, 127.7, 128.1, 128.6, 129.1(0), 129.1(3), 129.3, 129.8, 130.3, 132.1, 132.6, 135.4, 137.2, 138.1, 149.8, 154.4. m/z [HRMS-ESI⁺]: expected 625.2670 ([M+H]⁺), found: 625.2679 ([M+H]⁺).

### Preparation of SQF1321

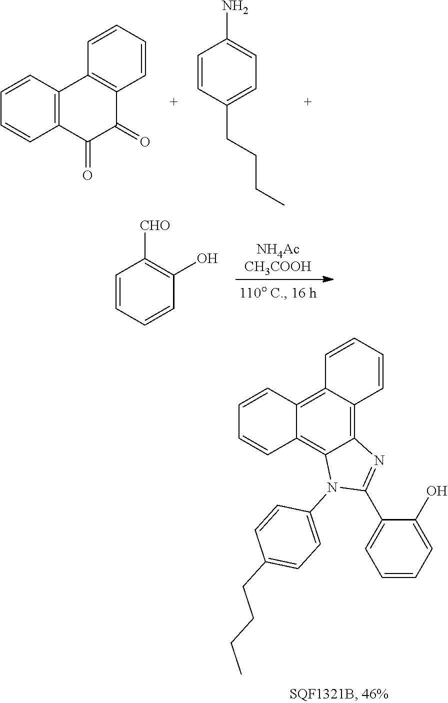 [SMILES: O=C1C(=O)c2ccccc2-c2ccccc21 ; CCCCc1ccc(N)cc1 ; O=Cc1ccccc1O ; CCCCc1ccc(-n2c(-c3ccccc3O)nc3c4ccccc4c4ccccc4c32)cc1]

A mixture of 9,10-phenanthrenequinone (2.08 g, 10 mmol), 4-n-butylaniline (2.37 mL, 15 mmol), salicylaldehyde (1.05 mL, 10 mmol), ammonium acetate (3.85 g, 50 mmol), and glacial acetic acid (60 mL) was stirred under argon in an oil bath held at 110° C. for 16 h. The mixture was allowed to cool to room temperature and then saturated potassium carbonate aqueous solution was added dropwise until pH=6. The mixture was then extracted using dichloromethane (3×50 mL). The combined organic portions were washed with brine (50 mL), dried over anhydrous magnesium sulfate, and filtered. The filtrate was collected and the solvent removed. The residue was purified by column chromatography over silica using dichloromethane:hexane (1:1-2:1) as eluent to give a white solid (2.05 g, 46%). mp 138-139° C. λₘₐₓ(dichloromethane)/nm: 256 sh (log ε/dm³ mol⁻¹ cm⁻¹ 4.63), 264 (4.77), 270 sh (4.61), 286 sh (4.23), 304 (4.16), 321 sh (4.27), 333 (4.39), 346 (4.29), 364 (4.32). λₘₐₓ(fluorescence) (dichloromethane)/nm: 471. ¹H NMR (δ, 500 MHz, CD₂Cl₂): 1.03 (3H, t, J=7.5, —CH₃), 1.48 (2H, sextet, J=7.5, —CH₂—), 1.80 (2H, m, —CH₂—), 2.89 (2H, t, J=7.5, —CH₂—), 6.52 (1H, ddd, J=1.5, J=7.0, J=8.5, O-Phenyl-H), 6.83 (1H, dd, J=1.5, J=8.0, O-Phenyl-H), 7.09 (1H, dd, J=1.5, J=8.5, O-Phenyl-H), 7.14 (1H, dd, J=1.0, J=8.5, Phenanthro-H), 7.22 (1H, ddd, J=1.5, J=7.5, J=8.5, O-Phenyl-H), 7.28 (1H, ddd, J=1.0, J=7.0, J=8.5, Phenanthro-H), 7.51-7.56 (5H, m, Phenanthro-H and N-Phenyl-H), 7.70 (1H, ddd, J=1.5, J=7.0, J=8.5, Phenanthro-H), 7.78 (1H, ddd, J=1.5, J=7.0, J=8.0, Phenanthro-H), 8.71 (1H, dd, J=1.0, J=8.0, Phenanthro-H), 8.73-8.75 (1H, m, Phenanthro-H), 8.78-8.80 (1H, m, Phenanthro-H), 13.72 (1H, brs, —OH). ¹³C NMR (δ, 125 MHz, CD₂Cl₂): 14.2, 22.6, 33.8, 35.8, 113.6, 118.2, 118.3, 121.5, 122.8, 123.1, 123.6, 124.4, 125.7, 126.1, 126.4, 126.7, 126.9, 127.6, 127.8, 128.8, 129.0, 129.7, 131.0, 121.2, 124.5, 136.7, 146.5, 149.0, 159.6. m/z [HRMS-ESI⁺]: expected 443.2118 ([M+H]⁺), found: 443.2117 ([M+H]⁺).

### Preparation of SQF1323

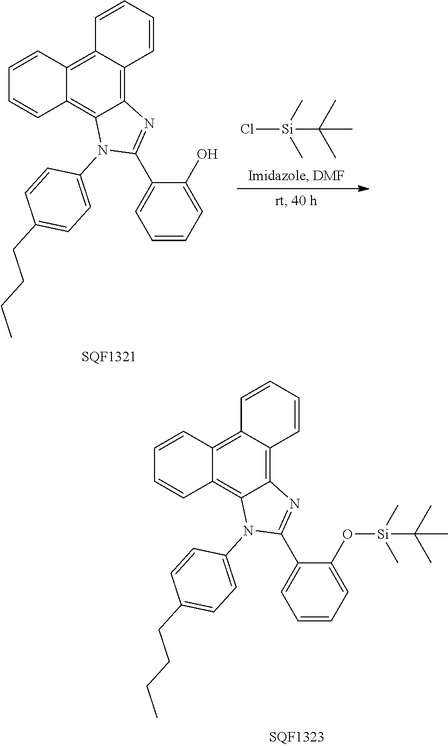

A mixture of SQF1321 (886 mg, 2.0 mmol), tert-butyldimethylsilyl chloride (362 mg, 2.4 mmol), imidazole (340 mg, 5.0 mmol), and anhydrous dimethylformamide (20 mL) was stirred under argon at room temperature for 40 h. Then water (50 mL) was added and the mixture was extracted with ethylacetate (3×50 mL) were added. The combined organic portions were washed with water (7×50 mL), dried over anhydrous magnesium sulfate, and filtered. The filtrate was collected and the solvent removed. The residue was purified by column chromatography over silica using dichloromethane:hexane (1:1-1:0) as eluent to give a white solid (831 mg, 75%). mp 102-103° C. λₘₐₓ(dichloromethane)/nm: 254 sh (log ε/dm³ mol⁻¹ cm⁻¹ 4.80), 260 (4.88), 284 (4.30), 295 sh (4.14), 308 (4.06), 324 sh (3.44), 342 (3.36), 357 (3.44). λₘₐₓ(fluorescence) (dichloromethane)/nm: 366, 382, 399 sh. ¹H NMR (δ, 500 MHz, CD₂Cl₂): 0.10 (6H, s, Si—CH₃), 0.71 (9H, s, Si—C(CH₃)₃), 0.96 (3H, t, J=7.5, —CH₃), 1.38 (2H, sextet, J=7.5, —CH₂—), 1.66 (2H, m, —CH₂—), 2.70 (2H, t, J=8.0, —CH₂—), 6.84 (1H, dd, J=1.0, J=8.5, O-Phenyl-H), 6.93 (1H, ddd, J=1.0, J=7.5, J=8.0, O-Phenyl-H), 7.21-7.29 (5H, m, O-Phenyl-H and Phenanthro-H and AA′BB′ of N-Phenyl-H), 7.32 (1H, dd, J=2.0, J=7.5, Phenanthro-H), 7.33-7.35 (2H, AA′BB′, N-Phenyl-H), 7.52 (1H, ddd, J=1.5, J=7.0, J=8.5, Phenanthro-H), 7.65 (1H, ddd, J=1.5, J=7.0, J=8.5, Phenanthro-H), 7.71 (1H, ddd, J=1.0, J=7.0, J=8.0, Phenanthro-H), 8.72-8.75 (2H, m, Phenanthro-H), 8.78-8.80 (1H, m, Phenanthro-H). ¹³C NMR (δ, 125 MHz, CD₂Cl₂): −4.4, 14.1, 18.2, 22.6, 25.5, 33.6, 35.6, 120.1, 121.0, 121.4, 122.8, 123.5(0), 123.5(2), 124.0, 124.3, 125.0, 125.5, 126.5, 127.5, 127.7, 128.0, 128.3, 128.5, 129.2, 129.6, 131.0, 132.5, 136.0, 137.5, 144.7, 150.2, 155.2. m/z [HRMS-ESI⁺]: expected 557.2983 ([M+H]⁺), found: 557.2987 ([M+H]⁺).

### Preparation of SQF1332

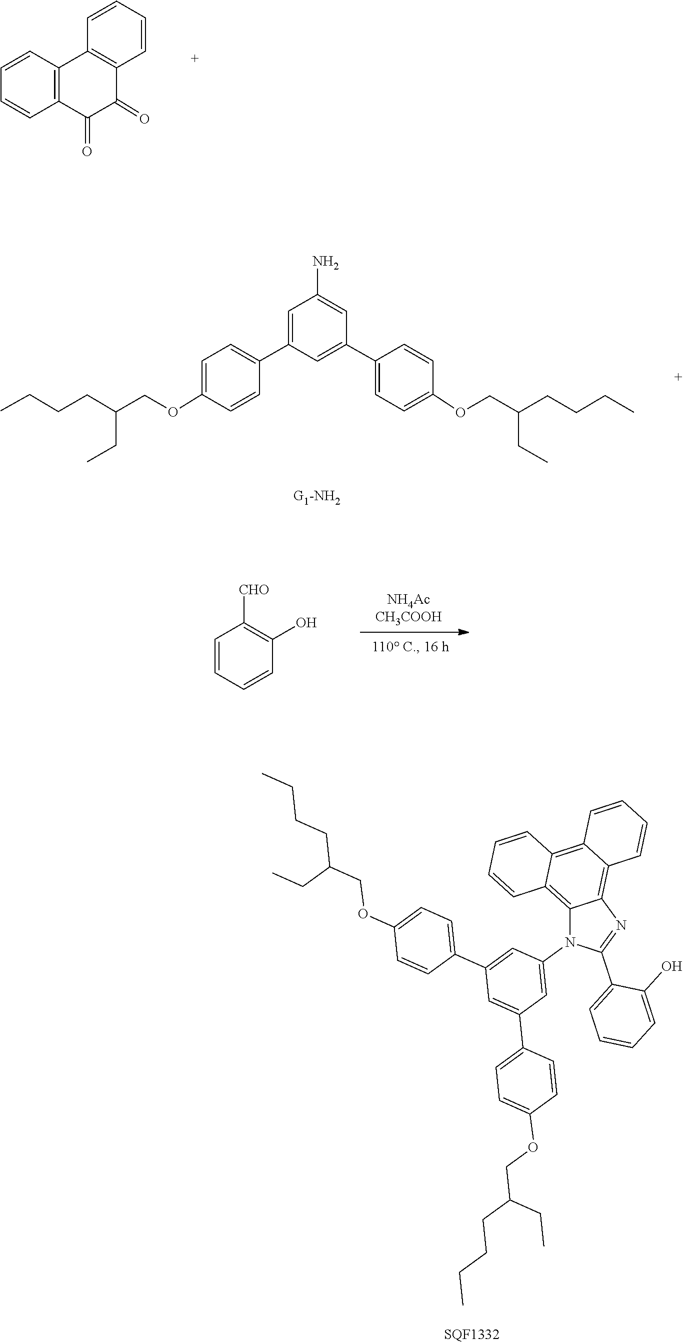

A mixture of 9,10-phenanthrenequinone (375 mg, 1.8 mmol), G₁-NH₂ (1030 mg, 2.0 mmol), salicylaldehyde (220 mg, 1.8 mmol), ammonium acetate (694 mg, 9 mmol), and glacial acetic acid (10 mL) was stirred under argon in an oil bath held at 110° C. for 16 h. The mixture was allowed to cool to room temperature and then water (50 mL) was added. Saturated potassium carbonate aqueous solution was added dropwise until pH=6. The mixture was then extracted using dichloromethane (3×50 mL). The combined organic portions were washed with brine (50 mL), dried over anhydrous magnesium sulfate, and filtered. The filtrate was collected and the solvent removed. The residue was purified by column chromatography over silica using dichloromethane:hexane (1:1) as eluent and then precipitation from dichloromethane/methanol to give a slightly yellowish solid (507 mg, 35%). mp 85-86° C. λₘₐₓ(dichloromethane)/nm: 257 sh (log ε/dm³ mol⁻¹ cm⁻¹ 4.80), 265 (4.95), 271 sh (4.92), 334 (4.39), 346 (4.31), 364 (4.33). λₘₐₓ(fluorescence) (dichloromethane)/nm: 453. ¹H NMR (δ, 500 MHz, CD₂Cl₂): 0.88-0.94 (12H, m, —CH₃), 1.27-1.55 (16H, m, —CH₂—), 1.73 (2H, septet, J=6.0, —CH), 3.86-3.91 (4H, m, O—CH₂), 6.55 (1H, ddd, J=1.5, J=7.5, J=8.5, O-Phenyl-H), 6.98-7.01 (4H, AA′BB′, G1-SPh-H), 7.06 (1H, dd, J=1.5, J=8.0, O-Phenyl-H), 7.10 (1H, dd, J=1.5, J=8.5, O-Phenyl-H), 7.21 (1H, ddd, J=1.5, J=7.0, J=8.5, O-Phenyl-H), 7.31 (1H, ddd, J=1.0, J=7.0, J=8.0, Phenanthro-H), 7.41 (1H, dd, J=1.0, J=8.5, Phenanthro-H), 7.55 (1H, ddd, J=1.5, J=7.0, J=8.5, Phenanthro-H), 7.64-7.67 (4H, AA′BB′, G1-SPh-H), 7.71 (1H, ddd, J=1.5, J=7.0, J=8.5, Phenanthro-H), 7.77 (2H, d, J=1.5, G1-BP—H), 7.79 (1H, ddd, J=1.0, J=7.0, J=8.0, Phenanthro-H), 8.18 (1H, t, J=1.5, G1-BP—H), 8.73 (1H, dd, J=1.5, J=8.0, Phenanthro-H), 8.76 (1H, d, J=8.5, Phenanthro-H), 8.81 (1H, d, J=8.0, Phenanthro-H), 13.75 (1H, brs, —OH). ¹³C NMR (δ, 125 MHz, CD₂Cl₂): 11.2, 14.2, 23.4, 24.2, 29.4, 30.8, 39.7, 71.0, 113.5, 115.4, 118.2, 118.5, 121.6, 122.8, 123.1, 123.7, 124.5, 124.9, 125.7, 126.2, 126.4, 126.8, 126.9, 127.2, 127.5, 127.9, 128.6, 128.8, 129.7, 131.1, 131.7, 134.7, 140.2, 144.3, 149.0, 159.6, 160.3. m/z [HRMS-ESI⁺]: expected 795.4520(100%), 796.4554(60%), 797.4587(17%) ([M+H]⁺), found: 795.4521(100%), 796.4558(70%), 797.4600(20%) ([M+H]⁺).

### Preparation of SQF1344

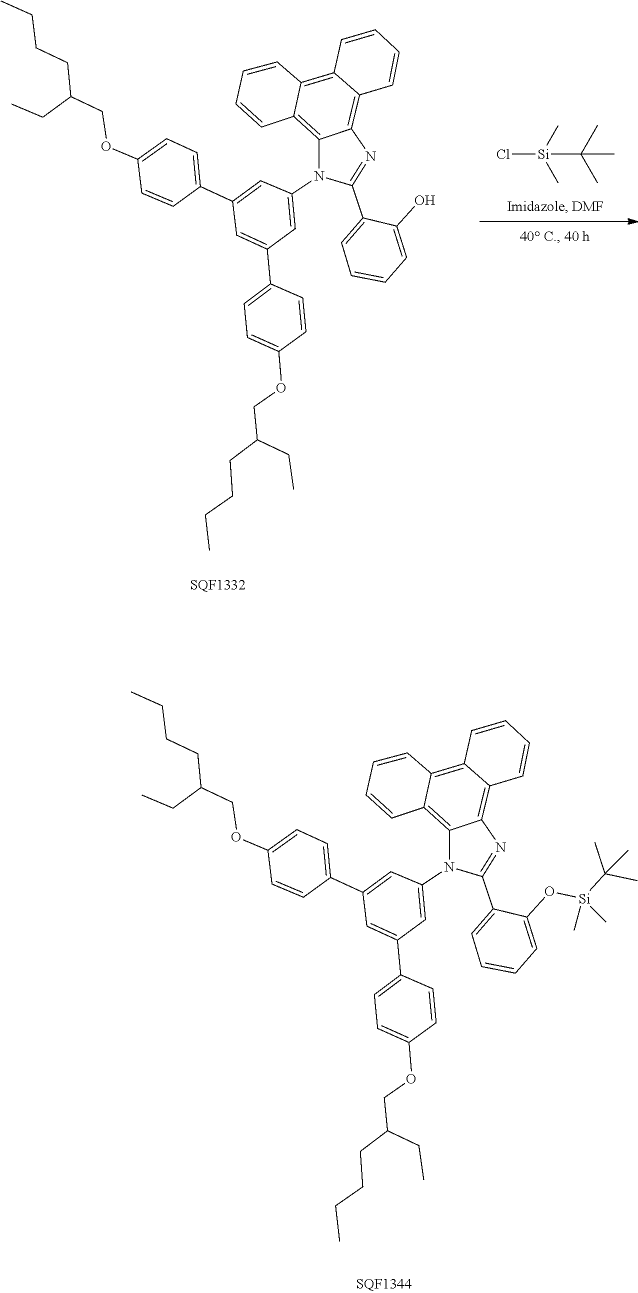

A mixture of SQF1332 (270 mg, 0.34 mmol), tert-butyldimethylsilyl chloride (201 mg, 1.30 mmol), imidazole (198 mg, 2.90 mmol), and anhydrous dimethylformamide (15 mL) was stirred under argon in an oil bath held at 40° C. for 44 h. Then water (50 mL) and ethylacetate (100 mL) were added and the mixture was separated. Then the organic layer was washed with water (6×50 mL), dried over anhydrous magnesium sulfate, and filtered. The filtrate was collected and the solvent removed. The residue was purified by column chromatography over silica using dichloromethane:hexane (1:1-1:0) as eluent to give a white solid (160 mg, 52%). mp 82-83° C. λₘₐₓ(dichloromethane)/nm: 260 (log ε/dm³ mol⁻¹ cm⁻¹ 5.00), 280 sh (4.82), 307 sh (4.30), 340 (3.38), 357 (3.43). λₘₐₓ(fluorescence) (dichloromethane)/nm: 365, 381, 399 sh. ¹H NMR (δ, 500 MHz, CD₂Cl₂): 0.11 (6H, s, Si—CH₃), 0.71 (9H, s, Si—C(CH₃)₃), 0.90-0.95 (12H, m, —CH₃), 1.30-1.56 (16H, m, —CH₂—), 1.74 (2H, septet, J=6.0, —CH), 3.86-3.91 (4H, m, O—CH₂), 6.92 (1H, dd, J=1.0, J=8.5, O-Phenyl-H), 6.94-6.97 (4H, AA′BB′, G1-SPh-H), 6.98 (1H, ddd, J=1.0, J=7.5, J=8.5, O-Phenyl-H), 7.30 (1H, ddd, J=2.0, J=7.5, J=8.5, O-Phenyl-H), 7.33 (1H, ddd, J=1.0, J=7.0, J=8.0, Phenanthro-H), 7.40 (1H, dd, J=2.0, J=7.5, 0-Phenyl-H), 7.49-7.52 (4H, AA′BB′, G1-SPh-H), 7.54 (1H, ddd, J=1.5, J=7.0, J=8.5, Phenanthro-H), 7.58 (1H, dd, J=1.0, J=8.5, Phenanthro-H), 7.61 (2H, d, J=1.5, G1-BP—H), 7.67 (1H, ddd, J=1.5, J=7.0, J=8.5, Phenanthro-H), 7.74 (1H, ddd, J=1.0, J=7.0, J=8.0, Phenanthro-H), 7.84 (1H, t, J=1.5, G1-BP—H), 8.75-8.79 (2H, m, Phenanthro-H), 8.82 (1H, d, J=8.5, Phenanthro-H). ¹³C NMR (δ, 125 MHz, CD₂Cl₂): −4.4, 11.2, 14.2, 18.2, 23.4, 24.2, 25.5, 29.4, 30.9, 39.8, 71.0, 115.2, 120.2, 121.3, 121.6, 122.9, 123.5, 124.2, 124.4, 124.6, 125.2, 125.5, 125.6, 126.7, 127.5(1), 127.5(4), 128.0, 128.5, 129.3, 131.2, 132.2, 132.4, 137.6, 139.3, 142.7, 150.3, 155.3, 160.0. m/z [HRMS-ESI⁺]: expected 909.5385(100%), 910.5419(66%), 911.5452(21%), 912.5486(4%) ([M+H]⁺), found: 909.5393(100%), 910.5419(71%), 911.5443(29%), 912.5462(7%) ([M+H]⁺).

### Preparation of SQF1348

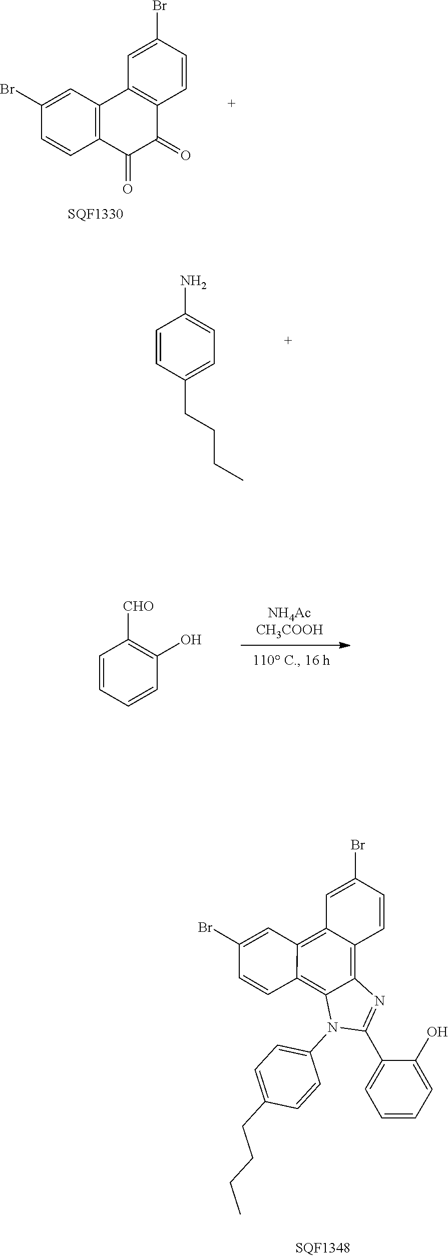 [SMILES: O=C1C(=O)c2ccc(Br)cc2-c2cc(Br)ccc21 ; CCCCc1ccc(N)cc1 ; O=Cc1ccccc1O ; CCCCc1ccc(-n2c(-c3ccccc3O)nc3c4ccc(Br)cc4c4cc(Br)ccc4c32)cc1]

A mixture of 3,6-dibromophenanthrene-9,10-dione (1.10 g, 3.0 mmol), 4-n-butylaniline (0.71 mL, 4.5 mmol), salicylaldehyde (0.32 mL, 3 mmol), ammonium acetate (1.16 g, 15 mmol), and glacial acetic acid (10 mL) was stirred under argon in an oil bath held at 110° C. for 16 h. The mixture was allowed to cool to room temperature and then saturated potassium carbonate aqueous solution was added dropwise until pH=6. The mixture was then extracted using dichloromethane (3×50 mL). The combined organic portions were washed with brine (50 mL), dried over anhydrous magnesium sulfate, and filtered. The filtrate was collected and the solvent removed. The residue was purified by column chromatography over silica using dichloromethane:hexane (1:10-1:1) as eluent to give a yellow solid (0.57 g, 32%). mp 227-228° C. λₘₐₓ(dichloromethane)/nm: 260 sh (log ε/dm³ mol⁻¹ cm⁻¹ 4.68), 267 (4.77), 276 sh (4.63), 295 (4.30), 304 (4.25), 326 sh (4.40), 339 (4.44), 359 sh (4.14), 378 (4.02). λₘₐₓ(fluorescence) (dichloromethane)/nm: 392, 408, 472. ¹H NMR (δ, 500 MHz, DMSO-d6): 0.94 (3H, t, J=7.5, —CH₃), 1.35 (2H, sextet, J=7.5, —CH₂—), 1.66 (2H, quintet, —CH₂—), 2.74 (2H, t, J=7.5, —CH₂—), 6.64 (1H, ddd, J=1.0, J=7.0, J=8.0, O-Phenyl-H), 6.91 (1H, d, J=9.0, Phenanthro-H), 6.92 (1H, dd, J=1.0, J=8.0, O-Phenyl-H), 7.01 (1H, dd, J=1.5, J=8.0, O-Phenyl-H), 7.23 (1H, ddd, J=1.5, J=7.5, J=8.5, O-Phenyl-H), 7.45-7.47 (2H, A′ABB′, N-Phenyl-H), 7.48 (1H, dd, J=2.0, J=9.0, Phenanthro-H), 7.53-7.55 (2H, A′ABB′, N-Phenyl-H), 7.93 (1H, dd, J=2.0, J=8.5, Phenanthro-H), 8.49 (1H, d, J=8.5, Phenanthro-H), 9.13-9.15 (2H, m, Phenanthro-H), 11.50 (1H, brs, —OH). ¹³C NMR (δ, 125 MHz, DMSO-d6): 13.8, 21.5, 32.7, 34.4, 115.1, 116.5, 118.2, 119.2, 119.6, 121.3, 121.9, 123.9, 125.0, 126.6, 126.7, 127.4, 128.3(0), 128.3(2), 129.0, 129.2, 130.0(8), 130.1, 131.1(0), 131.1(2), 134.7, 135.1, 144.8, 149.8, 157.2. m/z [HRMS-ESI⁺]: expected 599.0328, 601.0308, 603.0287 ([M+H]⁺), found: 599.0318, 601.0297, 603.0286 ([M+H]⁺).

### Preparation of SQF1350

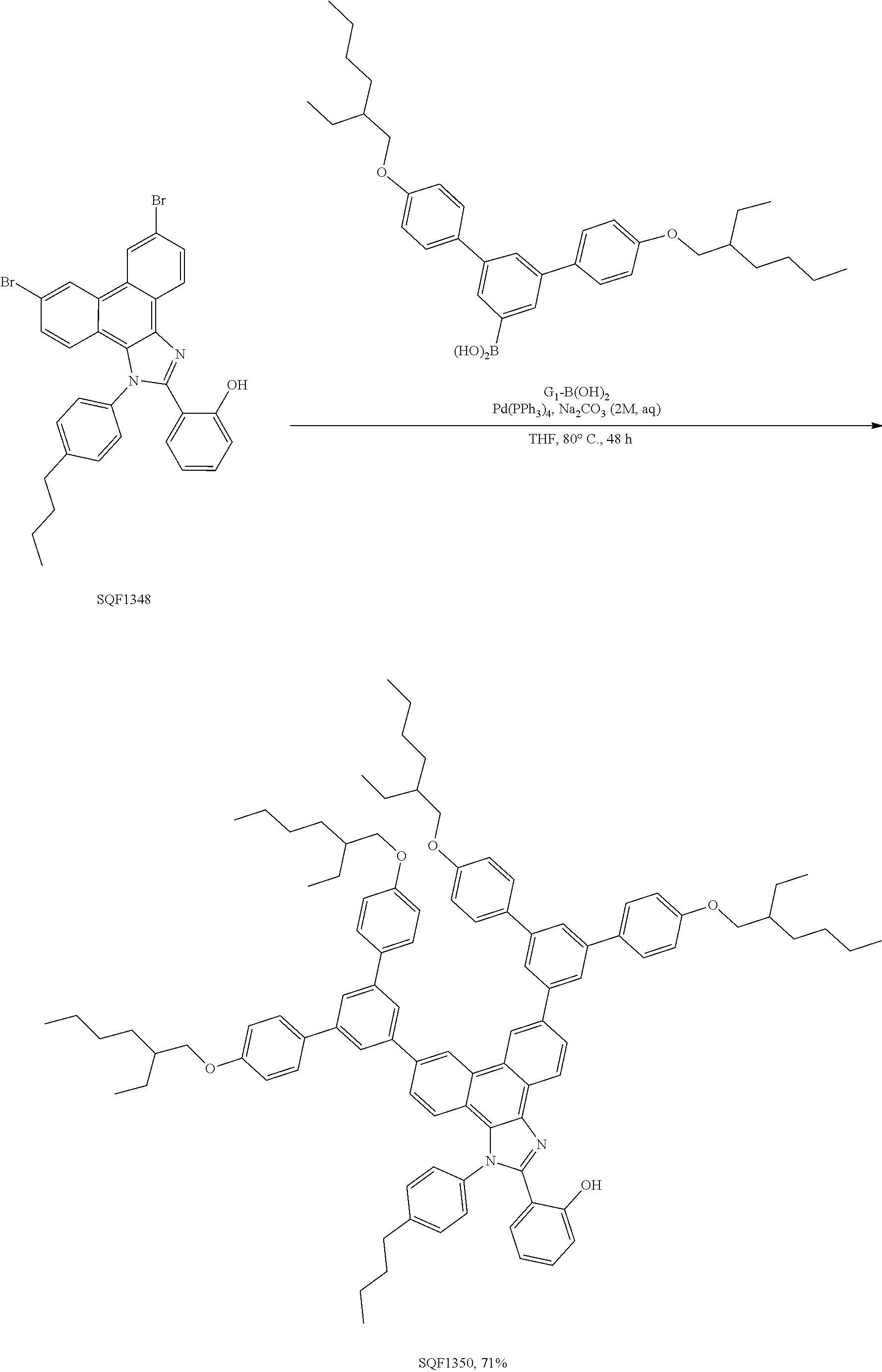

A mixture of SQF1348 (200 mg, 0.3 mmol), G₁-B(OH)₂ (530 mg, 1.0 mmol), sodium carbonate (aq. 2 M, 5 mL), and tetrahydrofuran (15 mL) was placed in a round-bottom flask, placed under vacuum and then backfilled with Ar(g) six times. Then tetrakis(triphenylphosphine)palladium(O) (38 mg, 0.03 mmol) was added and the resulting mixture was placed under vacuum and then backfilled with Ar(g) six times. The mixture was held in an oil bath at 80° C. for 48 h under argon protection. After cooling to room temperature, 50 mL of dichloromethane and 50 mL of water were added and the layers were separated. The aqueous phase was extracted with dichloromethane (2×50 mL), and the combined organic phases were washed with brine (2×50 mL), dried over anhydrous magnesium sulfate, filtered, and evaporated in vacuo. The residue was purified by column chromatography over silica using dichloromethane:hexane (1:1) as eluent to afford the product as a light white solid (332 mg, 71%). mp 102-103° C. λₘₐₓ(dichloromethane)/nm: 279 (log ε/dm³ mol⁻¹ cm⁻¹ 5.15), 344 (4.66), 359 sh (4.54), 378 (4.40). λₘₐₓ(fluorescence) (dichloromethane)/nm: 388, 408, 471. ¹H NMR (δ, 500 MHz, CD₂Cl₂): 0.90-0.97 (24H, m, EtHx-CH₃), 1.05 (3H, t, J=7.5, Bu-CH₃), 1.32-1.56 (34H, m, EtHx-CH₂ and Bu-CH₂), 1.74 (4H, septet, J=6.0, EtHx-CH), 1.79-1.85 (2H, m, Bu-CH₂), 2.91 (2H, t, J=7.5, Bu-CH₂), 3.86-3.93 (8H, m, O—CH₂), 6.54 (1H, ddd, J=1.5, J=8.0, J=8.5, O-Phenyl-H), 6.88 (1H, dd, J=1.5, J=8.0, O-Phenyl-H), 6.96-7.02 (8H, AA′BB′, G1-SPh-H), 7.11 (1H, dd, J=1.5, J=8.0, O-Phenyl-H), 7.24 (1H, ddd, J=1.5, J=7.0, J=8.5, O-Phenyl-H), 7.26 (1H, d, J=8.5, Phenanthro-H), 7.60 (4H, brs, N-Phenyl-H), 7.63-7.67 (5H, Phenanthro-H and AA′BB′ of G1-SPh-H), 7.69-7.72 (4H, AA′BB′, G1-SPh-H), 7.76 (1H, t, J=1.5, G1-BP—H), 7.80 (1H, t, J=1.5, G1-BP—H), 7.83 (2H, d, J=1.5, G1-BP—H), 7.95 (2H, d, J=1.5, G1-BP—H), 8.15 (1H, d, J=8.0, Phenanthro-H), 8.83 (1H, d, J=8.5, Phenanthro-H), 9.14 (1H, s, Phenanthro-H), 9.20 (1H, s, Phenanthro-H), 13.75 (1H, brs, —OH). ¹³C NMR (δ, 125 MHz, CD₂Cl₂): 11.3, 14.1(8), 14.2(5), 22.6, 23.5, 24.2, 29.5, 30.9, 33.9, 35.8, 39.8, 71.0, 113.6, 115.2(0), 115.2(2), 118.2, 118.3, 122.0, 122.4(8), 122.5(1), 123.1, 123.4, 124.5, 124.6, 124.8, 125.6, 126.5, 126.7, 127.6, 127.8, 128.5, 128.6, 129.1, 129.2, 130.2, 131.1, 131.3, 133.4, 133.6, 134.7, 136.7, 138.6, 139.5, 142.3(4), 142.3(9), 142.4(1), 142.9, 146.6, 149.3, 150.6(7), 159.7(1). m/z [HRMS-ESI⁺]: expected 1411.8801(93%), 1412.8834(100%), 1413.8868(53%) ([M+H]⁺), found: 1411.8784(75%), 1412.8825(100%), 1413.8850(50%) ([M+H]⁺).

### Preparation of SQF1352

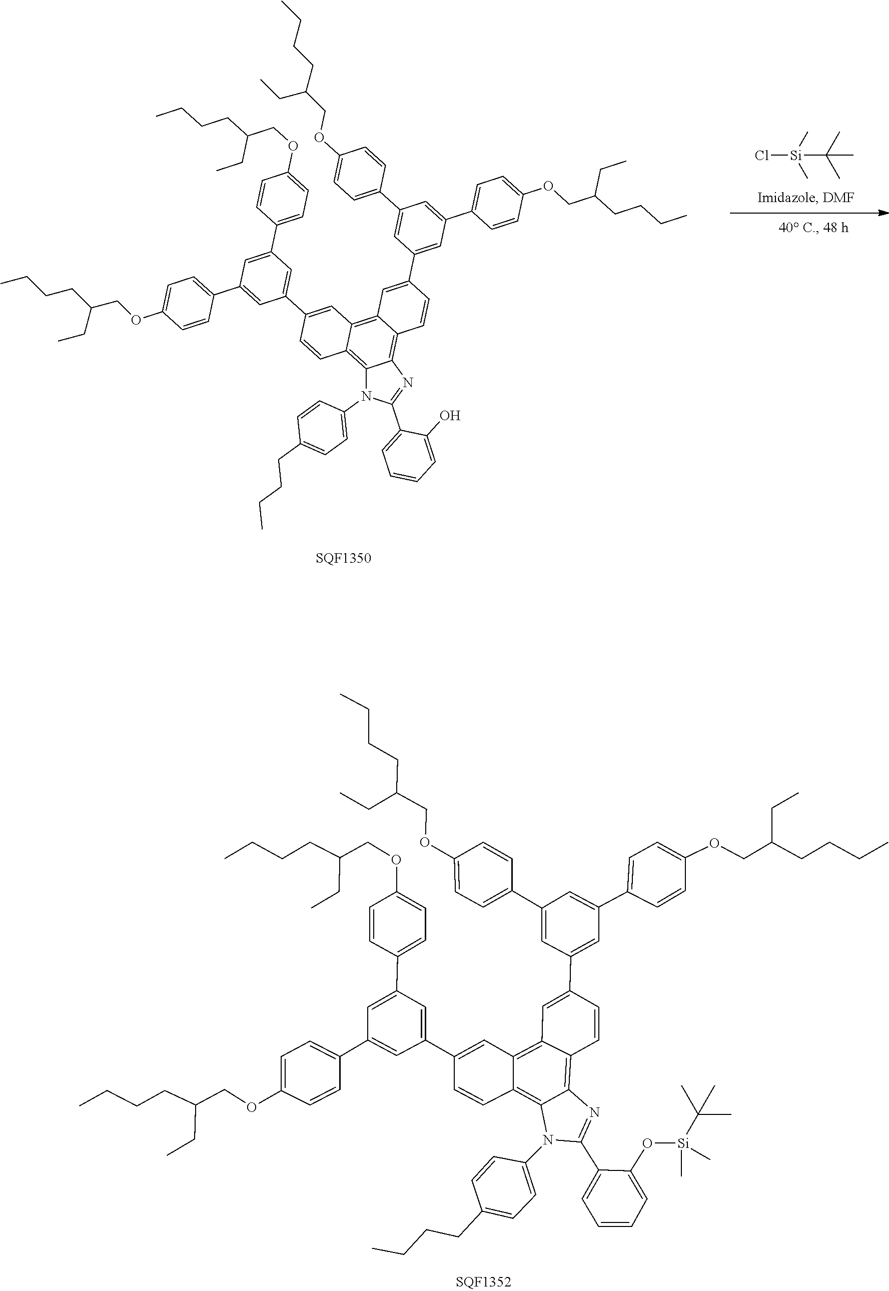

A mixture of SQF1350 (200 mg, 0.14 mmol), tert-butyldimethylsilyl chloride (201 mg, 1.30 mmol), imidazole (198 mg, 2.90 mmol), and anhydrous dimethylformamide (15 mL) was stirred under argon in an oil bath held at 40° C. for 48 h. Then water (50 mL) and ethylacetate (100 mL) were added and the mixture was separated. Then the organic layer was washed with water (5×50 mL), dried over anhydrous magnesium sulfate, and filtered. The filtrate was collected and the solvent removed. The residue was purified by column chromatography over silica using dichloromethane:hexane (1:1-1:0) as eluent and then precipitation from dichloromethane and methanol to give a white solid (177 mg, 82%). mp 101-102° C. λₘₐₓ(dichloromethane)/nm: 279 (log ε/dm³ mol⁻¹ cm⁻¹ 5.21), 324 sh (4.47), 370 sh (3.70). λₘₐₓ(fluorescence) (dichloromethane)/nm: 384, 401, 428 sh. ¹H NMR (δ, 500 MHz, CD₂Cl₂): 0.14 (6H, s, Si—CH₃), 0.75 (9H, s, Si—C(CH₃)₃), 0.90-0.98 (27H, m, EtHx-CH₃ and Bu-CH₃), 1.30-1.58 (34H, m, EtHx-CH₂ and Bu-CH₂), 1.65-1.69 (2H, m, Bu-CH₂), 1.74 (4H, septet, J=6.0, EtHx-CH), 2.72 (2H, t, J=8.0, Bu-CH₂), 3.86-3.93 (8H, m, O—CH₂), 6.86 (1H, dd, J=1.0, J=8.5, O-Phenyl-H), 6.94-7.02 (9H, m, O-Phenyl-H and AA′BB′ of G1-SPh-H), 7.27-7.32 (3H, m, O-Phenyl-H and AA′BB′ of N-Phenyl-H), 7.36 (1H, d, J=8.5, Phenanthro-H), 7.37 (1H, dd, J=2.0, J=7.5, O-Phenyl-H), 7.40-7.42 (2H, AA′BB′, N-Phenyl-H), 7.64-7.67 (5H, m, Phenanthro-H and AA′BB′ of G1-SPh-H), 7.69-7.72 (4H, AA′BB′, G1-SPh-H), 7.76 (1H, t, J=1.5, G1-BP—H), 7.80 (1H, t, J=1.5, G1-BP—H), 7.84 (2H, d, J=1.5, G1-BP—H), 7.96 (2H, d, J=1.5, G1-BP—H), 8.10 (1H, dd, J=1.5, J=8.5, Phenanthro-H), 8.88 (1H, d, J=8.5, Phenanthro-H), 9.15 (1H, d, J=1.5, Phenanthro-H), 9.20 (1H, d, J=1.5, Phenanthro-H). ¹³C NMR (δ, 125 MHz, CD₂Cl₂): −4.3, 11.3, 14.1, 14.23, 18.3, 22.7, 23.5, 24.2, 25.6, 29.5, 30.9, 33.7, 35.7, 39.8, 71.0, 115.2, 120.1, 121.1, 122.0, 122.3, 122.9(7), 123.0(2), 123.5, 123.9, 124.4, 124.5, 124.6, 124.8, 126.2, 127.4, 127.5, 128.0, 128.5(6), 128.6(2), 128.8, 129.7, 131.1, 132.5, 133.5, 133.6, 135.9, 137.6, 138.1, 138.7, 142.4, 142.7, 143.2, 144.8, 150.6, 155.2, 159.7. m/z [HRMS-ESI⁺]: expected 1525.9665(88%), 1526.9699(100%), 1527.9732(38%) ([M+H]⁺), found: 1525.9689(40%), 1526.9751(100%), 1527.9733(55%) ([M+H]⁺).

### Preparation of SQF1356

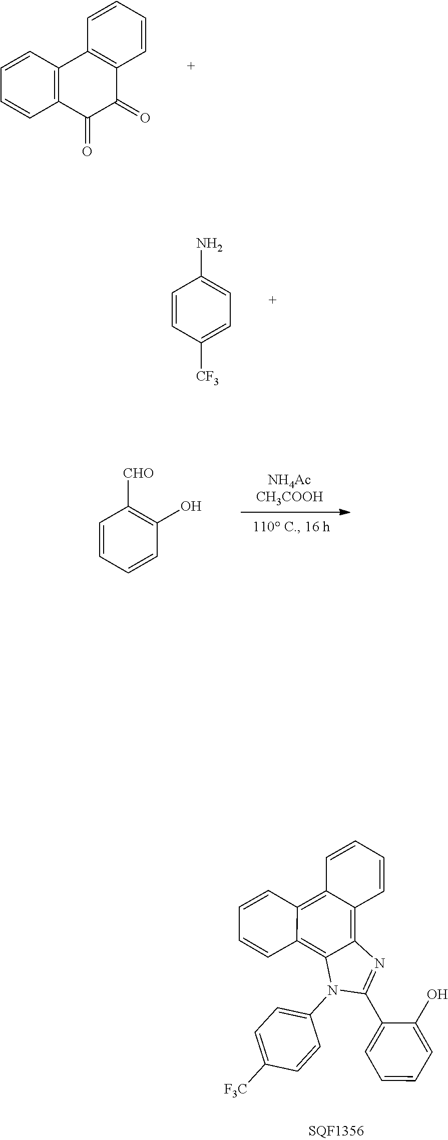 [SMILES: O=C1C(=O)c2ccccc2-c2ccccc21 ; Nc1ccc(C(F)(F)F)cc1 ; O=Cc1ccccc1O ; Oc1ccccc1-c1nc2c3ccccc3c3ccccc3c2n1-c1ccc(C(F)(F)F)cc1]

A mixture of 9,10-phenanthrenequinone (0.86 g, 4.1 mmol), 4-trifluoromethylaniline (1.00 g, 6.2 mmol), salicylaldehyde (0.43 mL, 4.1 mmol), ammonium acetate (1.60 g, 20.7 mmol), and glacial acetic acid (20 mL) was stirred under argon in an oil bath held at 110° C. for 16 h. The mixture was allowed to cool to room temperature and then saturated potassium carbonate aqueous solution was added dropwise until pH=6. The mixture was then extracted using dichloromethane (3×50 mL). The combined organic portions were washed with brine (50 mL), dried over anhydrous magnesium sulfate, and filtered. The filtrate was collected and the solvent removed. The residue was purified by column chromatography over silica using dichloromethane:hexane (1:1) as eluent to give a white solid (0.72 g, 38%). mp 186-187° C. λₘₐₓ(dichloromethane)/nm: 255 sh (log ε/dm³ mol-1 cm⁻¹ 4.63), 263 (4.76), 270 sh (4.61), 285 sh (4.28), 303 sh (4.15), 318 sh (4.12), 334 (4.35), 345 (4.32), 363 (4.33). λₘₐₓ(fluorescence) (dichloromethane)/nm: 486. ¹H NMR (δ, 500 MHz, CDCl₃): 6.54 (1H, ddd, J=1.5, J=7.0, J=8.5, O-Phenyl-H), 6.61 (1H, dd, J=1.5, J=8.0, O-Phenyl-H), 6.98 (1H, dd, J=1.0, J=8.5, Phenanthro-H), 7.15 (1H, dd, J=1.5, J=8.5, O-Phenyl-H), 7.24 (1H, ddd, J=1.5, J=7.0, J=8.5, O-Phenyl-H), 7.28 (1H, ddd, J=1.0, J=7.0, J=8.5, Phenanthro-H), 7.55 (1H, ddd, J=1.5, J=7.0, J=8.5, Phenanthro-H), 7.73 (1H, ddd, J=1.5, J=7.0, J=8.5, Phenanthro-H), 7.78 (1H, ddd, J=1.0, J=7.0, J=8.0, Phenanthro-H), 7.79-7.81 (4H, AA′BB′, N-Phenyl-H), 8.01-8.02 (4H, AA′BB′, N-Phenyl-H), 8.70-8.72 (2H, m, Phenanthro-H), 8.79 (1H, d, J=8.0, Phenanthro-H), 13.50 (1H, brs, —OH). ¹³C NMR (δ, 125 MHz, CDCl₃): 118.2(2), 118.2(4), 120.4, 122.2, 122.6, 123.2, 123.5 (q, ¹J_{C-F}=271.5), 124.4, 125.5, 125.6, 125.9, 126.3, 126.7, 126.8, 127.6, 127.9 (q, ³J_{C-F}=3.5), 128.4, 129.5, 129.9, 131.0, 132.7 (q, ²J_{C-F}=33.0), 134.8, 142.3, 148.3, 159.0. m/z [HRMS-ESI⁺]: expected 455.1366 ([M+H]⁺), found: 455.1370 ([M+H]⁺).

### Preparation of SQF1360

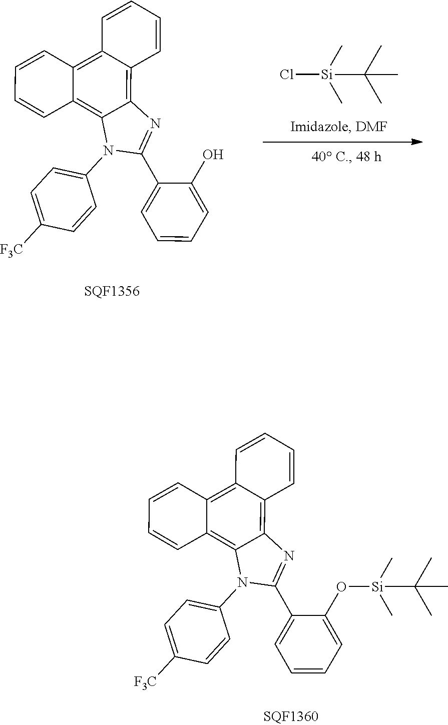

A mixture of SQF1356 (455 mg, 1.0 mmol), tert-butyldimethylsilyl chloride (452 mg, 3.0 mmol), imidazole (450 mg, 6.6 mmol), and anhydrous dimethylformamide (20 mL) was stirred under argon in an oil bath held at 40° C. for 44 h. Then water (50 mL) and ethylacetate (50 mL) were added. The organic phase was separated, washed with water (7×50 mL), dried over anhydrous magnesium sulfate, and filtered. The filtrate was collected and the solvent removed. The residue was purified by recrystallisation using dichloromethane/methanol to give a white crystal (361 mg, 64%). mp 186-187° C. ax(dichloromethane)/nm: 259 (log ε/dm³ mol⁻¹ cm⁻¹ 4.87), 282 sh (4.34), 306 (4.05), 340 (3.40), 355 (3.43). λₘₐₓ(fluorescence) (dichloromethane)/nm: 363, 380, 397. ¹H NMR (δ, 500 MHz, DMSO-d6): −0.09 (6H, s, Si—CH₃), 0.63 (9H, s, Si—C(CH₃)₃), 6.88 (1H, dd, J=1.0, J=8.5, O-Phenyl-H), 6.97 (1H, ddd, J=1.5, J=7.5, J=7.5, O-Phenyl-H), 7.08 (1H, dd, J=1.0, J=8.5, Phenanthro-H), 7.33 (1H, ddd, J=2.0, J=7.5, J=8.5, O-Phenyl-H), 7.40 (1H, ddd, J=1.0, J=7.0, J=8.5, Phenanthro-H), 7.43 (1H, dd, J=2.0, J=7.5, O-Phenyl-H), 7.58 (1H, ddd, J=1.5, J=7.0, J=8.5, Phenanthro-H), 7.69 (1H, ddd, J=1.5, J=7.0, J=8.5, Phenanthro-H), 7.72-7.74 (4H, AA′BB′, N-Phenyl-H), 7.76 (1H, ddd, J=1.0, J=7.0, J=8.0, Phenanthro-H), 7.94-7.96 (4H, AA′BB′, N-Phenyl-H), 8.62 (1H, dd, J=1.0, J=8.0, Phenanthro-H), 8.89 (1H, d, J=8.5, Phenanthro-H), 8.95 (1H, d, J=8.5, Phenanthro-H). ¹³C NMR (δ, 125 MHz, DMSO-d6): −4.7, 17.5, 25.1, 119.3, 120.0, 120.8, 121.9, 122.0, 122.4, 123.6, 123.7 (q, ¹J_{C-F}=271.0), 124.6, 125.2, 125.6, 126.4, 126.8, 127.4, 127.6, 128.4, 129.2, 129.7 (q, ²J_{C-F}=32.0), 131.3, 132.2, 136.7, 141.1, 149.3, 154.0. m/z [HRMS-ESI⁺]: expected 569.2231 ([M+H]⁺), found: 569.2236 ([M+H]⁺).

### Preparation of SQF1148

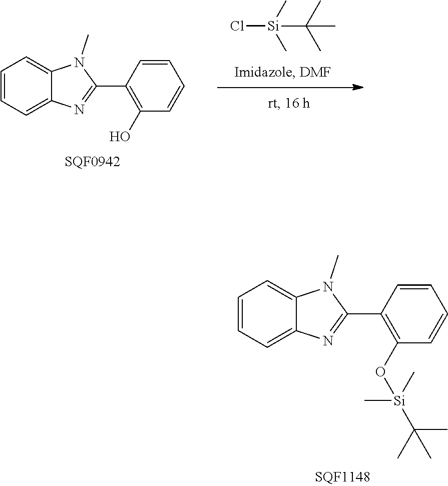

A mixture of SQF0942 (T. Shida, T. Mutai, K. Araki, CrystEngComm, 2013, 15, 10179-10182) (224 mg, 1.0 mmol), tert-butyldimethylsilyl chloride (181 mg, 1.2 mmol), imidazole (170 mg, 2.5 mmol), and anhydrous dimethylformamide (10 mL) was stirred under argon at room temperature for 16 h. Then water (50 mL) were added and the mixture was extracted with ethylacetate (5×50 mL). Then combined organic portions were washed with water (7×50 mL), dried over anhydrous sodium sulfate, and filtered. The filtrate was collected and the solvent removed. The residue was purified by column chromatography over neutral aluminium oxide using dichloromethane:ethylacetate (1:0-10:1) as eluent to give a white solid (307 mg, 91%). mp 93-94° C. ¹H NMR (δ, 500 MHz, CDCl₃): −0.09 (6H, s, —CH₃), 0.72 (9H, s, —C(CH₃)₃), 3.68 (3H, s, N—CH₃), 6.97 (1H, dd, J=1.0, J=8.0, O-Phenyl-H), 7.13 (1H, m, O-Phenyl-H), 7.27-7.33 (2H, m, Benzoimidazolyl-H), 7.37 (1H, m, Benzoimidazolyl-H), 7.41 (1H, m, O-Phenyl-H), 7.59 (1H, dd, J=1.5, J=7.5, O-Phenyl-H), 7.81 (1H, m, Benzoimidazolyl-H). ¹³CNMR (δ, 125 MHz, CDCl₃): −4.9, 17.8, 25.3, 30.8, 109.2, 119.7, 120.4, 121.9, 122.0, 122.4, 122.8, 131.3, 132.2, 135.9, 142.9, 152.3, 153.8. m/z [HRMS-ESI⁺]: expected 339.1887 ([M+H]⁺), found: 339.1899 ([M+H]⁺).

### Preparation of SQF1393

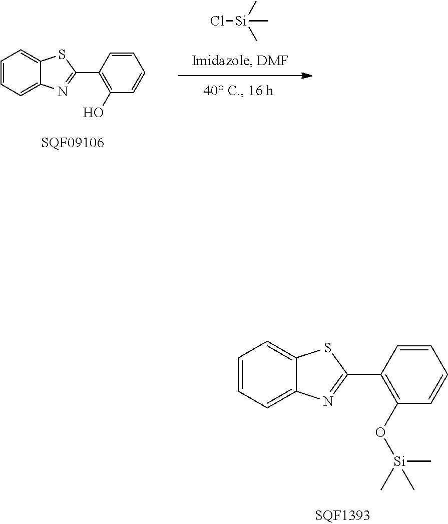

A mixture of SQF09106 (F. S. Santos, E. Ramasamy, V. Ramamurthy & F. S. Rodembusch, J. Mater. Chem. C, 2016, 4, 2820-2827.) (455 mg, 2.0 mmol), trimethylsilyl chloride (0.38 mL, 3.0 mmol), imidazole (450 mg, 6.6 mmol), and anhydrous dimethylformamide (10 mL) was stirred under argon at 40° C. for 16 h. Then ethylacetate (50 mL) and water (50 mL) were added and the mixture was separated. Then the organic layer was washed with water (5×50 mL), dried over anhydrous magnesium sulfate, and filtered. The filtrate was collected and the solvent removed. The residue was purified by recrystallisation in hexane to give a white solid (402 mg, 67%). mp 44-45° C. λₘₐₓ(dichloromethane)/nm: 250 (log ε/dm³ mol⁻¹ cm¹ 3.95), 258 (3.96), 289 sh (4.14), 298 (4.18), 310 sh (4.22), 321 (4.29), 331 sh (4.18). λₘₐₓ(fluorescence) (dichloromethane)/nm: 345, 361, 376, 399 sh. ¹H NMR (δ, 500 MHz, CDCl₃): 0.44 (9H, s, —CH₃), 6.99 (1H, dd, J=1.0, J=8.0, O-Phenyl-H), 7.13 (1H, ddd, J=1.0, J=7.0, J=8.0, O-Phenyl-H), 7.35-7.39 (2H, m, 0-Phenyl-H and Benzothiazolyl-H), 7.49 (1H, ddd, J=1.5, J=7.0, J=8.5, Benzothiazolyl-H), 7.93-7.95 (1H, m, Benzothiazolyl-H), 8.08-8.10 (1H, m, Benzothiazolyl-H), 8.49 (1H, dd, J=2.0, J=8.0, O-Phenyl-H). ¹³C NMR (δ, 125 MHz, CDCl₃): 0.70, 119.1, 121.2, 121.6, 122.8, 124.4, 124.6, 125.9, 129.8, 131.4, 135.9, 152.1, 153.6, 163.5. m/z [HRMS-ESI⁺]: expected 300.0873 ([M+H]⁺), found: 300.0886 ([M+H]⁺).

### Preparation of SQF1394

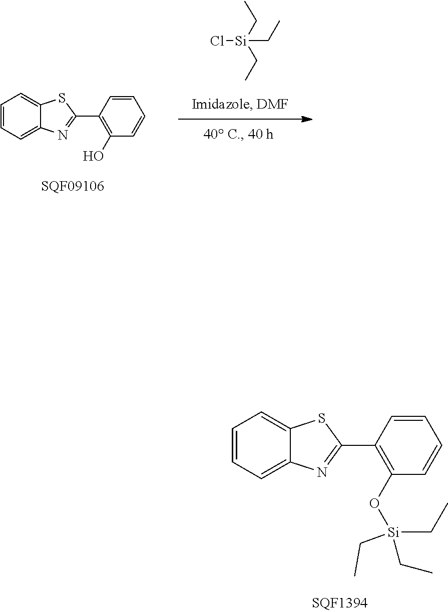

A mixture of SQF09106 (F. S. Santos, E. Ramasamy, V. Ramamurthy & F. S. Rodembusch, J. Mater. Chem. C, 2016, 4, 2820-2827.) (455 mg, 2.0 mmol), triethylsilyl chloride (0.50 mL, 3.0 mmol), imidazole (450 mg, 6.6 mmol), and anhydrous dimethylformamide (10 mL) was stirred under argon at 40° C. for 40 h. Then ethylacetate (50 mL) and water (50 mL) were added and the mixture was separated. Then the organic layer was washed with water (5×50 mL), dried over anhydrous magnesium sulfate, and filtered. The filtrate was collected and the solvent removed. The residue was purified by column chromatography over silica using dichloromethane:hexane (1:5-1:1) to give a semi-solid (629 mg, 94%). λₘₐₓ(dichloromethane)/nm: 250 (log ε/dm³ mol⁻¹ cm⁻¹ 3.96), 258 (3.97), 289 sh (4.14), 299 (4.18), 311 sh (4.22), 322 (4.29), 333 sh (4.17). λₘₐₓ(fluorescence) (dichloromethane)/nm: 346, 362, 377, 398 sh. ¹H NMR (δ, 500 MHz, CDCl₃): 0.92-0.97 (6H, m, —CH₂—), 1.02-1.05 (9H, m, —CH₃), 6.99 (1H, ddd, J=0.5, J=1.0, J=8.5, O-Phenyl-H), 7.11 (1H, ddd, J=1.0, J=7.5, J=8.0, O-Phenyl-H), 7.35 (1H, ddd, J=2.0, J=7.5, J=8.0, O-Phenyl-H), 7.37 (1H, ddd, J=1.0, J=7.0, J=8.0, Benzothiazolyl-H), 7.48 (1H, ddd, J=1.5, J=7.0, J=8.5, Benzothiazolyl-H), 7.93-7.95 (1H, m, Benzothiazolyl-H), 8.08-8.10 (1H, m, Benzothiazolyl-H), 8.48 (1H, ddd, J=0.5, J=2.0, J=8.0, O-Phenyl-H). ¹³C NMR (δ, 125 MHz, CDCl₃): 5.3, 6.6, 118.8, 121.2, 121.4, 122.7, 124.1, 124.5, 125.9, 129.8, 131.4, 136.0, 152.2, 154.0, 163.6. m/z [HRMS-ESI⁺]: expected 342.1342 ([M+H]⁺), found: 342.1338 ([M+H]⁺).

### Preparation of SQF1140

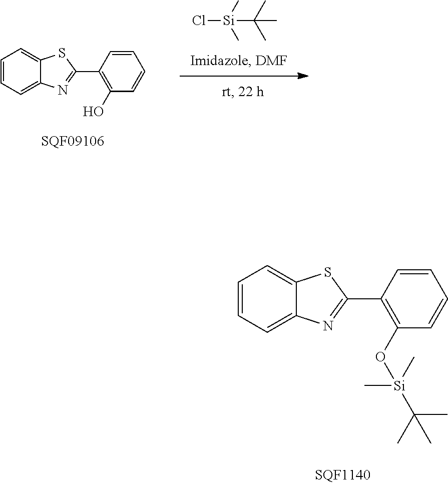

A mixture of SQF09106 (F. S. Santos, E. Ramasamy, V. Ramamurthy & F. S. Rodembusch, J. Mater. Chem. C, 2016, 4, 2820-2827.) (227 mg, 1.0 mmol), tert-butyldimethylsilyl chloride (181 mg, 1.2 mmol), imidazole (168 mg, 2.5 mmol), and anhydrous dimethylformamide (5 mL) was stirred under argon at room temperature for 22 h. Then ethylacetate (50 mL) were added and the mixture was washed with water (9×50 mL), dried over anhydrous sodium sulfate, and filtered. The filtrate was collected and the solvent removed. The residue was purified by column chromatography over silica using dichloromethane:hexane (1:3) to give a white solid (150 mg, 44%). mp 74-75° C. λₘₐₓ(dichloromethane)/nm: 250 (log ε/dm³ mol-1 cm¹ 3.96), 258 (3.96), 289 sh (4.14), 299 (4.17), 312 sh (4.21), 322 (4.26), 334 sh (4.12). λₘₐₓ(fluorescence) (dichloromethane)/nm: 346, 362, 377, 400 sh. ¹H NMR (δ, 500 MHz, CDCl₃): 0.39 (6H, s, —CH₃), 1.02 (9H, s, —C(CH₃)₃), 7.01 (1H, dd, J=1.0, J=8.5, O-Phenyl-H), 7.10 (1H, ddd, J=1.0, J=7.5, J=8.0, O-Phenyl-H), 7.34 (1H, ddd, J=2.0, J=7.5, J=8.5, O-Phenyl-H), 7.38 (1H, ddd, J=1.0, J=7.0, J=8.0, Benzothiazolyl-H), 7.49 (1H, ddd, J=1.0, J=7.0, J=8.5, Benzothiazolyl-H), 7.94 (1H, d, J=8.0, Benzothiazolyl-H), 8.09 (1H, d, J=8.0, Benzothiazolyl-H), 8.39 (1H, dd, J=2.0, J=8.0, O-Phenyl-H). ¹³C NMR (δ, 125 MHz, CDCl₃): −3.3, 18.9, 26.2, 119.5, 121.3, 121.4, 122.8, 124.4, 124.6, 125.9, 130.1, 131.3, 135.9, 152.3, 154.0, 163.8. m/z [HRMS-ESI⁺]: expected 342.134 ([M+H]⁺), found: 342.135 ([M+H]⁺).

### Preparation of SQF1395

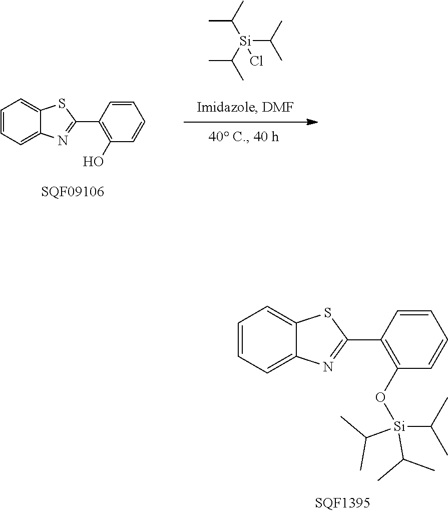 [SMILES: CC(C)[Si](Cl)(C(C)C)C(C)C ; Oc1ccccc1-c1nc2ccccc2s1 ; CC(C)[Si](Oc1ccccc1-c1nc2ccccc2s1)(C(C)C)C(C)C]

A mixture of SQF09106(F. S. Santos, E. Ramasamy, V. Ramamurthy & F. S. Rodembusch,. J. Mater. Chem. C, 2016, 4, 2820-2827.) (455 mg, 2.0 mmol), triisopropylsilyl chloride (0.65 mL, 3.0 mmol), imidazole (450 mg, 6.6 mmol), and anhydrous dimethylformamide (10 mL) was stirred under argon at 40° C. for 40 h. Then ethylacetate (50 mL) and water (50 mL) were added and the mixture was separated. Then the organic layer was washed with water (5×50 mL), dried over anhydrous magnesium sulfate, and filtered. The filtrate was collected and the solvent removed. The residue was purified by column chromatography over silica using dichloromethane:hexane (1:5-1:1) to give a white solid (707 mg, 92%). mp 107-108° C. λₘₐₓ(dichloromethane)/nm: 250 (log ε/dm³ mol⁻¹ cm⁻¹ 3.96), 258 (3.96), 289 sh (4.14), 300 (4.17), 312 sh (4.20), 323 (4.26), 335 sh (4.13). λₘₐₓ(fluorescence) (dichloromethane)/nm: 349, 363, 378, 402 sh. ¹H NMR (δ, 500 MHz, CDCl₃): 1.17 (18H, d, J=7.5, —CH₃), 1.51 (3H, septet, J=7.5, —CH), 7.00 (1H, dd, J=1.0, J=8.5, O-Phenyl-H), 7.09 (1H, ddd, J=1.0, J=7.5, J=8.0, O-Phenyl-H), 7.33 (1H, ddd, J=2.0, J=7.5, J=8.5, O-Phenyl-H), 7.37 (1H, ddd, J=1.0, J=7.0, J=8.0, Benzothiazolyl-H), 7.49 (1H, ddd, J=1.5, J=7.0, J=8.5, Benzothiazolyl-H), 7.93-7.95 (1H, m, Benzothiazolyl-H), 8.08-8.10 (1H, m, Benzothiazolyl-H), 8.48 (1H, ddd, J=0.5, J=2.0, J=8.0, O-Phenyl-H). ¹³C NMR (δ, 125 MHz, CDCl₃): 13.6, 18.0, 119.1, 121.2, 121.3, 122.8, 124.0, 124.5, 125.8, 130.1, 131.3, 136.0, 152.3, 154.3, 163.7. m/z [HRMS-ESI⁺]: expected 384.1812 ([M+H]⁺), found: 384.1806 ([M+H]⁺).

### Preparation of SQF1396

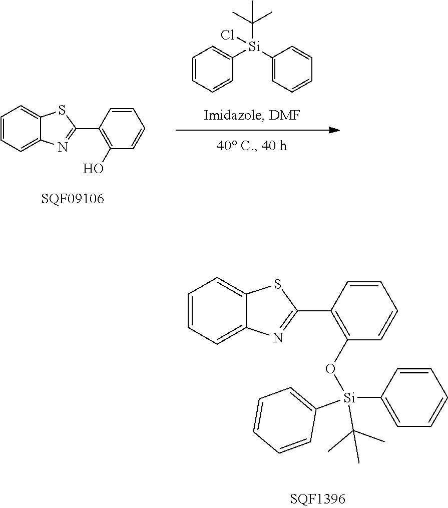 [SMILES: CC(C)(C)[Si](Cl)(c1ccccc1)c1ccccc1 ; Oc1ccccc1-c1nc2ccccc2s1 ; CC(C)(C)[Si](Oc1ccccc1-c1nc2ccccc2s1)(c1ccccc1)c1ccccc1]

A mixture of SQF09106 (F. S. Santos, E. Ramasamy, V. Ramamurthy & F. S. Rodembusch, J. Mater. Chem. C, 2016, 4, 2820-2827.) (455 mg, 2.0 mmol), tert-butyldiphenylsilyl chloride (0.78 mL, 3.0 mmol), imidazole (450 mg, 6.6 mmol), and anhydrous dimethylformamide (10 mL) was stirred under argon at 40° C. for 40 h. Then ethylacetate (50 mL) and water (50 mL) were added and the mixture was separated. Then the organic layer was washed with water (5×50 mL), dried over anhydrous magnesium sulfate, and filtered. The filtrate was collected and the solvent removed. The residue was purified by column chromatography over silica using dichloromethane:hexane (1:5-1:1) to give a semi-solid (855 mg, 92%). λₘₐₓ(dichloromethane)/nm: 258 (logs/dm³ mol⁻¹ cm⁻¹ 4.05), 275 sh (4.09), 289 sh (4.22), 301 sh (4.25), 311 sh (4.28), 321 (4.31), 332 sh (4.16). λₘₐₓ(fluorescence) (dichloromethane)/nm: 344, 360, 375, 396 sh. ¹H NMR (δ, 500 MHz, CDCl₃): 1.14 (9H, s, —CH₃), 6.55-6.57 (1H, m, O-Phenyl-H), 6.93-7.00 (2H, m, O-Phenyl-H), 7.36-7.46 (7H, m, Si-Phenyl-H and Benzothiazolyl-H), 7.52 (1H, ddd, J=1.5, J=7.5, J=8.5, Benzothiazolyl-H), 7.76-7.78 (4H, m, Si-Phenyl-H), 7.96-7.98 (1H, m, Benzothiazolyl-H), 8.12-8.14 (1H, m, Benzothiazolyl-H), 8.29 (1H, ddd, J=0.5, J=2.0, J=7.5, O-Phenyl-H). ¹³C NMR (δ, 125 MHz, CDCl₃): 19.4, 26.5, 120.5, 121.3, 122.9, 124.0, 124.7, 126.0, 127.9, 130.1, 130.4, 130.9, 132.1, 135.4, 136.0, 152.6, 153.6, 163.8. m/z [HRMS-ESI⁺]: expected 466.1655 ([M+H]⁺), found: 466.1645 ([M+H]⁺).

### Preparation of SQF13108

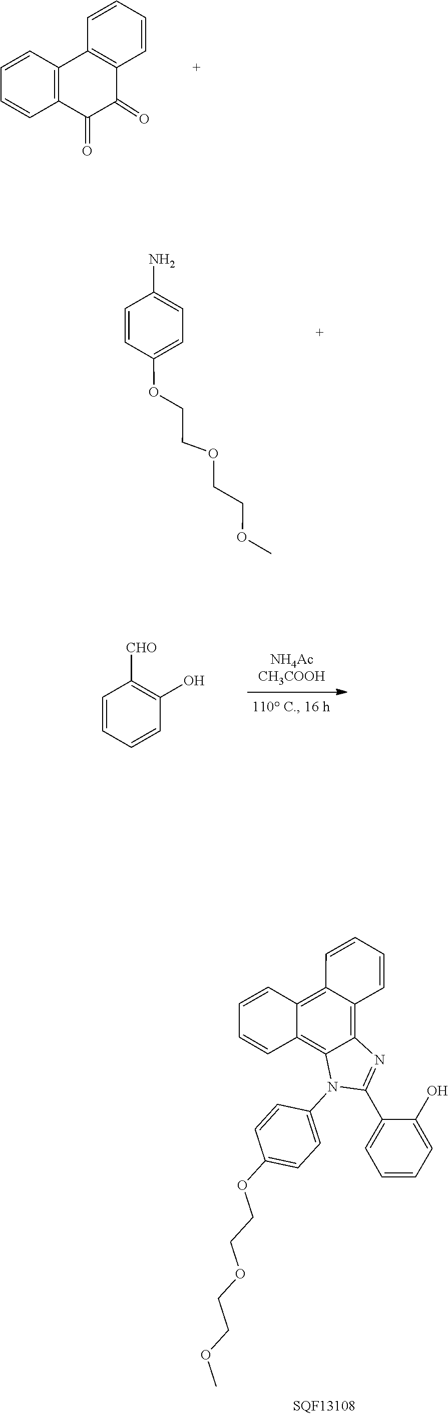 [SMILES: O=C1C(=O)c2ccccc2-c2ccccc21 ; COCCOCCOc1ccc(N)cc1 ; O=Cc1ccccc1O ; COCCOCCOc1ccc(-n2c(-c3ccccc3O)nc3c4ccccc4c4ccccc4c32)cc1]

A mixture of 9,10-phenanthrenequinone (1.04 g, 5.0 mmol), 4-[2-(2-methoxyethoxy)ethoxy]benzenamine [M. Stein, S. I. Middendorp, V. Carta, E. Pejo, D. E. Raines, S. A. Forman, E. Sigel, D. Trauner, Angew. Chem. Int. Ed. 2012, 51, 10500-10504] (1.58 g, 7.5 mmol), salicylaldehyde (0.52 mL, 5.0 mmol), ammonium acetate (1.90 g, 25.0 mmol), and glacial acetic acid (20 mL) was stirred under argon in an oil bath held at 110° C. for 16 h. The mixture was allowed to cool to room temperature and then saturated potassium carbonate aqueous solution was added dropwise until pH=6. The mixture was then extracted using dichloromethane (3×50 mL). The combined organic portions were washed with brine (50 mL), dried over anhydrous magnesium sulfate, and filtered. The filtrate was collected and the solvent removed. The residue was purified by column chromatography over silica using dichloromethane:hexane (1:1-1:0) and then dichloromethane:ethyl acetate (20:1) as eluent to give a yellowish solid (1.02 g, 40%). ¹H NMR (δ, 500 MHz, CD₂Cl₂): 3.39 (3H, s, —CH₃), 3.60 (2H, m, —CH₂), 3.74 (2H, m, —CH₂), 3.94 (2H, m, —CH₂), 4.31 (2H, m, —CH₂), 6.57 (1H, ddd, J=1.5, J=7.0, J=8.5, O-Phenyl-H), 6.89 (1H, dd, J=1.5, J=8.0, O-Phenyl-H), 7.08 (1H, dd, J=1.5, J=8.5, O-Phenyl-H), 7.19-7.27 (4H, m, O-Phenyl-H, Phenanthro-H and AA′BB′ of N-Phenyl-H), 7.33 (1H, ddd, J=1.0, J=7.0, J=8.5, Phenanthro-H), 7.52-7.57 (3H, m, Phenanthro-H and AA′BB′ of N-Phenyl-H), 7.70 (1H, ddd, J=1.5, J=7.0, J=8.5, Phenanthro-H), 7.77 (1H, ddd, J=1.5, J=7.0, J=8.0, Phenanthro-H), 8.68 (1H, dd, J=1.0, J=8.0, Phenanthro-H), 8.74 (1H, d, J=8.0, Phenanthro-H), 8.79 (1H, d, J=8.5, Phenanthro-H), 13.77 (1H, brs, —OH). ¹³C NMR (δ, 125 MHz, CD₂Cl₂): δ9.1, 68.4, 69.9, 71.1, 72.3, 113.6, 116.8, 118.1, 118.4, 121.4, 122.7, 123.1, 123.6, 124.4, 125.6, 126.1, 126.3, 126.6, 127.0, 127.7, 127.8, 128.7, 129.7, 130.4, 131.0, 131.8, 134.5, 149.2, 159.6, 160.7.

### Preparation of SQF13111

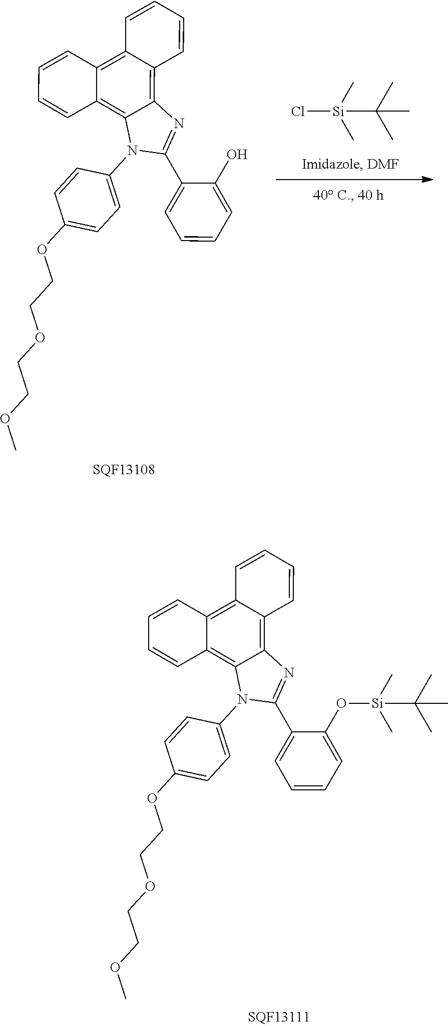

A mixture of SQF13108 (504 mg, 1.0 mmol), tert-butyldimethylsilyl chloride (226 mg, 1.5 mmol), imidazole (224 mg, 3.3 mmol), and anhydrous dimethylformamide (10 mL) was stirred under argon in an oil bath held at 40° C. for 40 h. Then water (50 mL) and ethylacetate (50 mL) was added. The organic phase was separated, washed with water (5×50 mL), dried over anhydrous magnesium sulfate, and filtered. The filtrate was collected and the solvent removed. The residue was purified by column chromatography over BioBeads using toluene as eluent to give a white solid (418 mg, 68%). ¹H NMR (δ, 500 MHz, CD₂Cl₂): 0.09 (6H, s, Si—CH₃), 0.71 (9H, s, Si—C(CH₃)₃), 3.39 (3H, s, —CH₃), 3.54 (2H, m, —CH₂), 3.68 (2H, m, —CH₂), 3.84 (2H, m, —CH₂), 4.15 (2H, m, —CH₂), 6.84 (1H, dd, J=1.0, J=8.5, O-Phenyl-H), 6.93-6.98 (3H, m, O-Phenyl-H and AA′BB′ of N-Phenyl-H), 7.25-7.33 (4H, m, O-Phenyl-H and Phenanthro-H), 7.34-7.37 (2H, AA′BB′, N-Phenyl-H), 7.52 (1H, ddd, J=2.0, J=6.0, J=8.5, Phenanthro-H), 7.64 (1H, ddd, J=1.5, J=7.0, J=8.0, Phenanthro-H), 7.71 (1H, ddd, J=1.0, J=7.0, J=8.0, Phenanthro-H), 8.72-8.74 (2H, m, Phenanthro-H), 8.79 (1H, d, J=8.5, Phenanthro-H). ¹³C NMR (δ, 125 MHz, CD₂Cl₂): −4.4, 18.2, 25.5, 59.1, 68.1, 69.9, 71.1, 72.3, 115.2, 120.1, 121.1, 121.3, 122.8, 122.4(6), 123.5(4), 124.0, 124.3, 125.0, 125.5, 126.6, 127.5, 127.8, 128.0, 128.3, 129.2, 129.9, 131.0, 131.2, 132.4, 137.4, 150.4, 155.1, 159.5.

Film preparation: The sensor compound was dissolved in toluene (10 mg/mL). Thin films were prepared on fused silica substrates by spin-coating at 2000 rpm for 60 s to give a thickness of 20-40 nm.

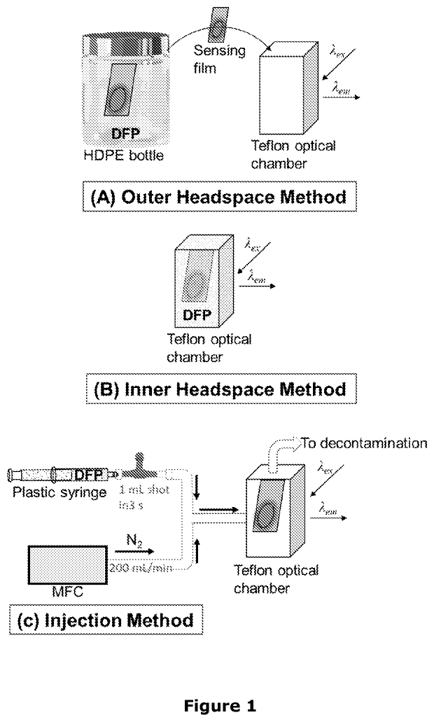

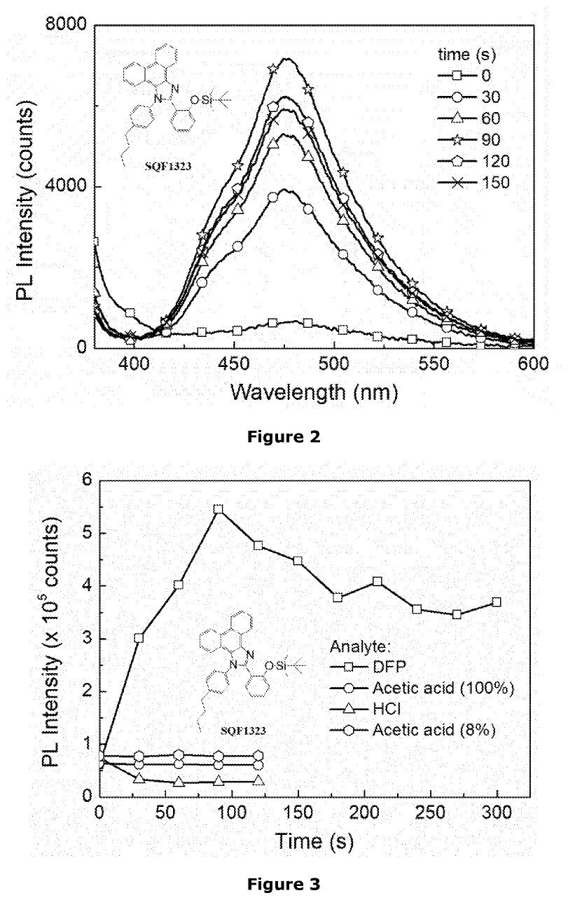

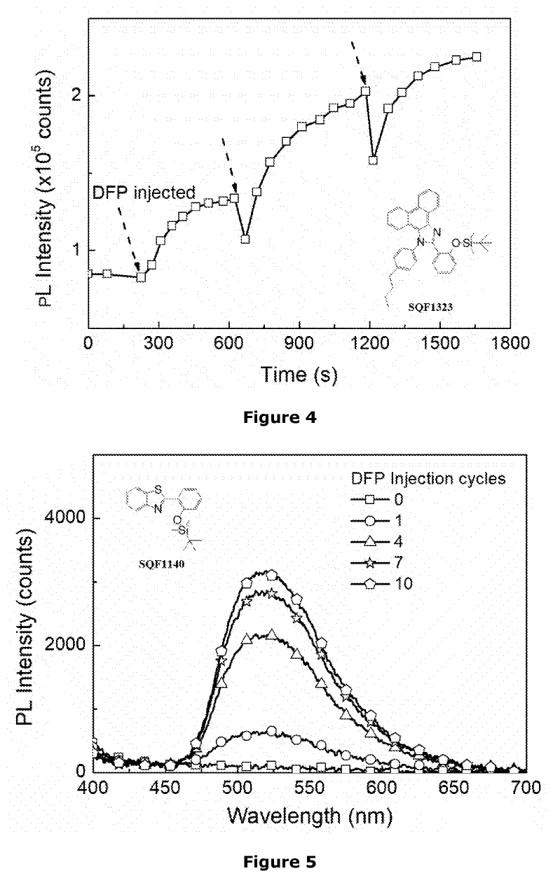

Vapour generation: Three methods (FIG. 1) were employed for the generation of DFP vapours. Caution: Dialkylfluorophosphates are highly toxic compounds. They should only be synthesized, purified, used and disposed of after rigorous risk assessment. All processes should only be undertaken by trained persons using appropriate personal protection equipment. Disclaimer: Work with Schedule 1 CWAs should only be performed at OPCW declared facilities.

Method A: 2 μL of DFP was added to a 200 mL HDPE plastic bottle and kept for 30 min to allow the analyte evaporation at 20-22° C. Sensing films were placed in the analyte-containing plastic bottle for a certain time and then moved to the optical chamber for PL spectra and kinetics measurements under ambient atmosphere.

Method B: 2 μL of DFP was added on the surface of a Teflon lid which was placed at the bottom of the optical chamber or a pipette droplet of sarin was added directly into the bottom of the chamber. Evaporation at 20-22° C. gave the analyte vapour. Sensing films were placed in the same chamber for PL spectra and kinetics measurements.

Method C: 2 μL of DFP was added to a plastic syringe and kept for 30 min to allow the analyte evaporation at 20-22° C. The vapour was then injected manually at a flow rate of 1 mL vapour per 3 s to a nitrogen flow (200 mL/min) and the mixed gas was introduced into the optical chamber for the sensing measurement. The nitrogen stream was eventually passed through a scrubbing solution (20 wt % sodium hydroxide in water) to break down the excess simulant.

Sensing measurements: The sensing film samples on fused silica substrates were mounted in a closed sample chamber which was connected via optical fibres to an LED light source (365 nm, OceanOptics) and a spectrometer (Flame, OceanOptics). The sample chamber featured three optical windows to allow for excitation of the films and subsequent detection of the film PL at right angles to the excitation. Film PL spectra before and after exposure to analyte and PL kinetics at the emissive peak were recorded through OceanView software (OceanOptics).

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

1. A method for detecting a nerve agent in an analyte, said nerve agent having a phosphorus-fluorine bond, which method comprises:
(a) contacting the analyte with a solid state composition comprising a sensor compound;
wherein the sensor compound comprises a basic nitrogen atom and a hydroxyaryl moiety protected by a silyl protecting group; and wherein, in the presence of hydrogen fluoride, said silyl group is cleaved to effect deprotection of the hydroxyl group thus forming a luminescent reporter compound;
(b) irradiating the solid state composition at a predetermined wavelength;
(c) measuring the luminescence to determine if the reporter compound is present; and
(d) determining whether the nerve agent is present in the analyte based on the measurement obtained in step (c).

2. A method according to claim 1, wherein the sensor compound has the Formula (IA): 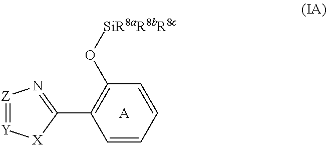
wherein:
A is an optionally substituted aryl group;
X is NR, Y is CR³, and Z is CR⁴;
X is S, Y is CR³, and Z is CR⁴;
X is O, Y is CR³, and Z is CR⁴;
X is NR, Y is CR³, and Z is NR;
X is O, Y is CR³, and Z is N;
R is H, optionally substituted aryl or optionally substituted alkyl;
R³ is H, optionally substituted aryl or optionally substituted alkyl;
R⁴ is H, optionally substituted aryl or optionally substituted alkyl;
or R³ and R⁴, together with the carbon atoms to which they are attached, form a cyclic moiety, for example an optionally substituted aryl moiety;
or
Formula (IB): 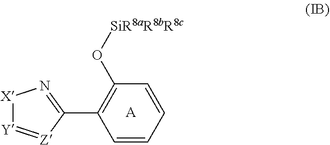
wherein:
X′ is O, Y′ is CR³ and Z′ is CR⁴;
X′ is S, Y′ is CR³ and Z′ is CR⁴; or
X′ is NR, Y′ is CR³ and Z′ is CR⁴; and
A, R, R³, R⁴, R^{8a}, R^{8b} and R^{8c} are as defined for Formula (IA).

3. The method according to claim 1, wherein the method is for vapour phase detection.

4. The method according to claim 1, wherein the method is for detection of contamination on a surface.

5. A sensing device for detection of a nerve agent in an analyte, said nerve agent having a P—F bond, the sensing device comprising:
A composition comprising a solid state sensor compound wherein the sensor compound comprises a basic nitrogen atom and a hydroxyaryl moiety protected by a silyl protecting group; and wherein, in the presence of hydrogen fluoride, said silyl group is cleaved to de-protect the hydroxyl group thus forming a luminescent reporter compound;
an irradiation source for irradiating the reporter compound with stimulating radiation at a predetermined wavelength;
a detector for measuring luminescence of the optical sensing element;
means for relating to an operator the luminescence measured by the detector; and
means for delivering the analyte for contacting with the sensor compound.

6. The sensing device according to claim 5, wherein the solid state sensor compound is comprised in a film, a coating on a substrate, a swab or an optical sensing element.

7. An optical sensing element for detection of a nerve agent having a phosphorus-fluorine bond, wherein said sensing element comprises a compound comprising a basic nitrogen atom and a hydroxyaryl moiety protected by a silyl protecting group; and wherein, in the presence of hydrogen fluoride, said silyl group is cleaved to de-protect the hydroxyl group thus forming a luminescent reporter compound.

8. A sensor compound of Formula (IA), (IB), (IC), (ID), (IE) or (IF).

9. A sensor compound of Formula (IA): 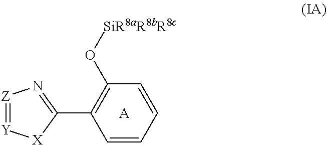
wherein:
A is an optionally substituted aryl group;
X is NR, Y is CR³, and Z is CR⁴;
X is S, Y is CR³, and Z is CR⁴;
X is O, Y is CR³, and Z is CR⁴;
X is NR, Y is CR³, and Z is NR;
X is O, Y is CR³, and Z is N;
R is H, optionally substituted aryl or optionally substituted alkyl;
R³ is H, optionally substituted aryl or optionally substituted alkyl;
R⁴ is H, optionally substituted aryl or optionally substituted alkyl;
or R³ and R⁴, together with the carbon atoms to which they are attached, form a cyclic moiety, for example an optionally substituted aryl moiety;

10. A sensor compound according to claim 9 selected from SQF1148, SQF1323, SQF1360, SQF1370, SQF1382, SQF1388, SQF1389, SQF1399, SQF13100, SQF13111, SQF1344, SQF1352, SQF1140, SQF1393, SQF1394, SQF1395, and SQF1396.

11. Use of a compound according to claim 8 for detecting a nerve agent, wherein said nerve agent has a phosphorus-fluorine bond.

12. The use of claim 11, wherein the nerve agent is a G-series nerve agent, preferably Sarin, Cyclosarin or Soman.
